(19) United States
(12) Patent Application Publication
(10) Pub. No.: US 2025/0210128 A1
(43) Pub. Date: Jun. 26, 2025

(54) COMPUTER-IMPLEMENTED DESIGN OF PEPTIDE:RECEPTOR SIGNALING COMPLEXES FOR ENHANCED CHEMOTAXIS

(71) Applicant: Ecole Polytechnique Federale de Lausanne (EPFL), Lausanne (CH)

(72) Inventors: Patrick Barth, Lausanne (CH); Robert Jefferson, Lausanne (CH)

(21) Appl. No.: 18/852,201

(22) PCT Filed: Mar. 28, 2023
(86) PCT No.: PCT/EP2023/057928

§ 371 Date: Sep. 27, 2024

(30) Foreign Application Priority Data
Mar. 30, 2022 (EP) 22165413.0

(51) Int. Cl. G16B 15/30; C07K 14/52; C07K 14/715; C12N 5/0783

(52) U.S. Cl. CPC G16B 15/30; C07K 14/522; C07K 14/7158; C12N 5/0636

(57) ABSTRACT

The present invention relates to a computer-implemented method for engineering the interaction between a protein and a cognate peptide that are capable of forming a molecular complex, wherein the method comprises (a) preparing in silico a library of test peptides based on the cognate peptide, and the molecular complex of the protein and the cognate peptide; and/or (a′) preparing in silico a library of test protein scaffolds based at least on the parts of the protein that participate in forming the molecular complex with the cognate peptide; (b) docking in silico (i) the library of test peptides onto the library of protein scaffolds by modelling peptide-protein molecular complexes, or (ii) the cognate peptide on the library of protein scaffolds, or the library of test peptides onto the protein or the parts of the protein that participate in forming the molecular complex with the cognate peptide by modelling peptide-protein molecular complexes; (c) identifying the test peptides in the library and/or the protein scaffolds in the library for which low interface energy peptide-protein molecular complexes can be modelled, preferably by a combination of flexible peptide docking and/or de novo protein structure building; (d) identifying the interacting amino acids of the test peptides and/or the protein scaffolds in the ensemble of low interface energy modelled peptide-protein molecular complexes of step (c); (e) selecting and substituting in silico one or more of the interacting amino acids of the peptides and/or the protein scaffolds as identified in step (d) based on the ensemble of low interface energy peptide-protein molecular complexes as identified in step (c); and (f) generating in silico based on the peptides with substituted amino acid(s) and/or the protein scaffolds with substituted amino acid(s) of (e) an ensemble of peptides with substituted amino acid(s) and/or the protein scaffolds with substituted amino acid(s) for which the lowest interface energy peptide-protein molecular complexes can be modelled, thereby obtaining engineered peptides and/or proteins being capable of forming a molecular complex with engineered binding characteristics.

The present invention relates to a computer-implemented method for engineering the interaction between a protein and a cognate peptide that are capable of forming a molecular complex, wherein the method comprises (a) preparing in silico a library of test peptides based on the cognate peptide, and/or the molecular complex of the protein and the cognate peptide; and (a′) preparing in silico a library of test protein scaffolds based at least on the parts of the protein that participate in forming the molecular complex with the cognate peptide; (b) docking in silico (i) the library of test peptides onto the library of protein scaffolds by modelling peptide-protein molecular complexes, or (ii) the cognate peptide on the library of protein scaffolds, or the library of test peptides onto the protein or the parts of the protein that participate in forming the molecular complex with the cognate peptide by modelling peptide-protein molecular complexes; (c) identifying the test peptides in the library and/or the protein scaffolds in the library for which low interface energy peptide-protein molecular complexes can be modelled, preferably by a combination of flexible peptide docking and/or de novo protein structure building; (d) identifying the interacting amino acids of the test peptides and/or the protein scaffolds in the ensemble of low interface energy modelled peptide-protein molecular complexes of step (c); (e) selecting and substituting in silico one or more of the interacting amino acids of the peptides and/or the protein scaffolds as identified in step (d) based on the ensemble of low interface energy peptide-protein molecular complexes as identified in step (c); and (f) generating in silico based on the peptides with substituted amino acid(s) and/or the protein scaffolds with substituted amino acid(s) of (e) an ensemble of peptides with substituted amino acid(s) and/or the protein scaffolds with substituted amino acid(s) for which the lowest interface energy peptide-protein molecular complexes can be modelled, thereby obtaining engineered peptides and/or proteins being capable of forming a molecular complex with engineered binding characteristics.

Protein-peptide interactions play an important role in major cellular processes, and are associated with several human diseases. To understand and potentially regulate these cellular function and diseases it is important to know the molecular details of the interactions. However, because of peptide flexibility and the transient nature of protein-peptide interactions, peptides are difficult to study experimentally.

In particular, designing biosensors with arbitrary input and output behaviors is a grand challenge of synthetic biology. Current approaches focus on engineering binding to structurally well-defined protein¹ and small-molecule chemical cues², and couple molecular recognition to synthetic optical reporters that are built-in modular biosensor scaffolds. While this strategy provides elegant solutions to the design of in vitro diagnostics, applications for in vivo detection and synthetic cell biology rely on coupling the molecular sensor to the precise activation and orchestration of complex intracellular signaling functions that often cannot be recapitulated de novo. Harnessing synthetic sensing to fine-tuned native signaling functions in a biosensor scaffold is limited by the poor mechanistic understanding of allosteric signal transduction and lack of techniques to rationally engineer these properties.

Peptides mediate close to 40% of cell signaling functions through ubiquitous interactions with membrane receptors and soluble proteins^{3,4}. Unbound peptide ligands are often partially disordered in solution, which challenges structure determination, and computational sampling of the vast conformational space.

In contrast to rigid protein binders and small-molecule ligands, structural information on peptide binding is scarce and limits supervised training and validation of deep-learning⁵⁻⁷ and physics-based⁸ protein:peptide complex structure prediction approaches. The specific receptor:peptide engineering problem is further complicated by the high flexibility of both receptor and peptide ligand which through mutual induced fit often adopt a new conformation together to reach the active state and initiate signal transduction. The rational design of peptide-sensing receptors has not been reported to date.

Thus, further sophisticated computational methods for predicting structural information about protein-peptide interactions are needed.

Hence, the present invention relates in a first aspect to a computer-implemented method for engineering the interaction between a protein and a cognate peptide that are capable of forming a molecular complex, wherein the method comprises (a) preparing in silico a library of test peptides based on the cognate peptide, and/or the molecular complex of the protein and the cognate peptide; and (a′) preparing in silico a library of test protein scaffolds based at least on the parts of the protein that participate in forming the molecular complex with the cognate peptide; (b) docking in silico (i) the library of test peptides onto the library of protein scaffolds by modelling peptide-protein molecular complexes, or (ii) the cognate peptide on the library of protein scaffolds, or the library of test peptides onto the protein or the parts of the protein that participate in forming the molecular complex with the cognate peptide by modelling peptide-protein molecular complexes; (c) identifying the test peptides in the library and/or the protein scaffolds in the library for which low interface energy peptide-protein molecular complexes can be modelled, preferably by a combination of flexible peptide docking and/or de novo protein structure building; (d) identifying the interacting amino acids of the test peptides and/or the protein scaffolds in the ensemble of low interface energy modelled peptide-protein molecular complexes of step (c); (e) selecting and substituting in silico one or more of the interacting amino acids of the peptides and/or the protein scaffolds as identified in step (d) based on the ensemble of low interface energy peptide-protein molecular complexes as identified in step (c); and (f) generating in silico based on the peptides with substituted amino acid(s) and/or the protein scaffolds with substituted amino acid(s) of (e) an ensemble of peptides with substituted amino acid(s) and/or the protein scaffolds with substituted amino acid(s) for which the lowest interface energy peptide-protein molecular complexes can be modelled, thereby obtaining engineered peptides and/or proteins being capable of forming a molecular complex with engineered binding characteristics.

A computer-implemented method as used herein designates a method which involves the use of a computer, computer network or other programmable apparatus, where one or more features are realised wholly or partly by means of a computer program. Since the method is a computer-implemented method, also a computer-implemented program is described herein that when being executed on a computer causes the computer to carry out the method of the first aspect of the invention.

The result of the engineered interaction between a protein and a cognate peptide that are capable of forming a molecular complex is preferably a higher binding sensitivity and/or when being complexed a potent biological activity as illustrated by allosteric signal transduction responses across the cell membrane (when being tested in vivo or in vitro). Hence, a molecular complex with engineered binding characteristics preferably display (i) a binding sensitivity between the engineered peptides and/or proteins as compared to the non-engineered “base” molecular complex and/or (i) more potent biological activity as illustrated by allosteric signal transduction responses across the cell membrane (when being tested in vivo or in vitro) as compared to the non-engineered “base” molecular complex.

The term “protein” as used herein interchangeably with the term “polypeptide” describes molecular chains of amino acids, preferably linear molecular chains of amino acids including single chain proteins or their fragments, containing at least 100 amino acids. The term “peptide” as used herein describes a group of molecules consisting of up to 99 amino acids, preferably up to 75 amino acids and most preferably up to 50 amino acids. The term “peptide” as used herein describes a group of molecules consisting with increased further preference of at least 5 amino acids or at least 10 amino acids. The upper and lower lengths for the peptide may be combined into the respective ranges. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. Furthermore, peptidomimetics of such proteins/(poly)peptides where amino acid(s) and/or peptide bond(s) have been replaced by functional analogues are also encompassed by the invention. Such functional analogues include all known amino acids other than the 20 gene-encoded amino acids, such as selenocysteine. The terms “(poly)peptide” also refer to naturally modified (poly)peptides where the modification is effected e.g. by glycosylation, acetylation, phosphorylation and similar modifications which are well known in the art.

The term “molecular complex” as used herein designates an interaction between a protein and a cognate peptide that results in a stable association in which these two molecules are in close proximity to each other. It is formed when atoms or molecules bind together by sharing of electrons. It often, but not always, involves some chemical bonding. In a molecular complex the forces holding the components together are generally non-covalent, and thus are normally energetically weaker than covalent bonds.

The nature of the protein and the cognate peptide that are capable of forming a molecular complex are not particularly limited as long as they can bind to each other and thereby form a molecular complex. Protein-peptide interactions play a fundamental role in a wide variety of biological processes, such as cell signaling, regulatory networks, immune responses, and enzyme inhibition. Peptide bonds act in cell signaling and as immune modulators, among other important functions. In addition, protein-protein interactions are a fundamental part of all major biological processes. A particularly interesting class of protein-protein interactions are those involving interaction including intrinsically disordered regions. These regions are often the size of small peptide fragments 5 to 25 residues long and part of proteins involved in regulation, recognition, and signaling requiring dynamic and specific responses. When investigating these interactions, it is common practice to isolate the binding motif of the disordered region and analyze the binding as a protein-peptide interactions; see, for example, Akhe et al. (2019), Scientific Reports, 9:4267. Such protein-peptide interactions also envisioned herein.

The nature of the protein and the cognate peptide are in general a naturally occurring protein and cognate peptide that are known to form a molecular complex in vivo, preferably within an animal, and most preferably within a human.

The protein may be chosen, for example, from membrane receptors and soluble proteins, such as enzymes (e.g. tyrosine kinases), transporters or channels. Also immunoglobulins, such as antibodies or MHC complexes are envisioned. Many cognate peptides of such proteins are known in the art.

In order for the complex to be stable, the free energy of the complex by definition must be lower than the solvent separated molecules. It follows that the lower interface energy of a protein-peptide molecular complex is the more stable in said complex. Interface energy can be measured accurately and can also be calculated from molecular simulations (i.e. in silico modelling of molecular complexes); see, for example, Wu and Firoozabadi (2021), J. Phys. Chem., 25(26):5841-5848.

The term “in silico” as used herein refers to steps of the method of the invention that are performed on or with the aid of a computer or via computer simulation. The phrase is pseudo-Latin for ‘in silicon’ (in Latin it would be in silicio), referring to silicon in computer chips.

According to steps (a) and (a′) of the method of the invention (i) a library of test peptides based on the cognate peptide and/or the molecular complex of the protein and the cognate peptide; or (ii) a library of test protein scaffolds based at least on the parts of the protein that participate in forming the molecular complex with the protein, or both of these libraries are prepared in silico. The preparation of both libraries is mandatory, so that the method of the invention comprises steps (a) and (a′).

In silico libraries of protein scaffolds and/or peptides that are capable of forming a molecular complex can be built directly from the protein and peptide sequence databases and/or the conformation of the molecular complex; see, for example Hong and Kim (2016), Bioinformatics, 32(11):1709-1715. As for the members of the library it is possible to generate hundreds of thousands of tertiary structures for a given amino-acid sequence, known as decoys, in a few hours; see Akhater et al. (2020) BMC Bioinformatics, 21: 189 (2020). The members of the libraries are therefore also designated decoys herein.

In the examples herein below a library of protein scaffolds based on the CXCR4 receptors with 1000 decoys and a library of peptides based on CXCL12 with 10000 decoys were generated. Each library comprises in accordance with the invention independently with increasing preference at least 10 decoys, at least 100 decoys, at least 500 decoys, at least 1000 decoys, at least 5000 decoys, and at least 10000 decoys.

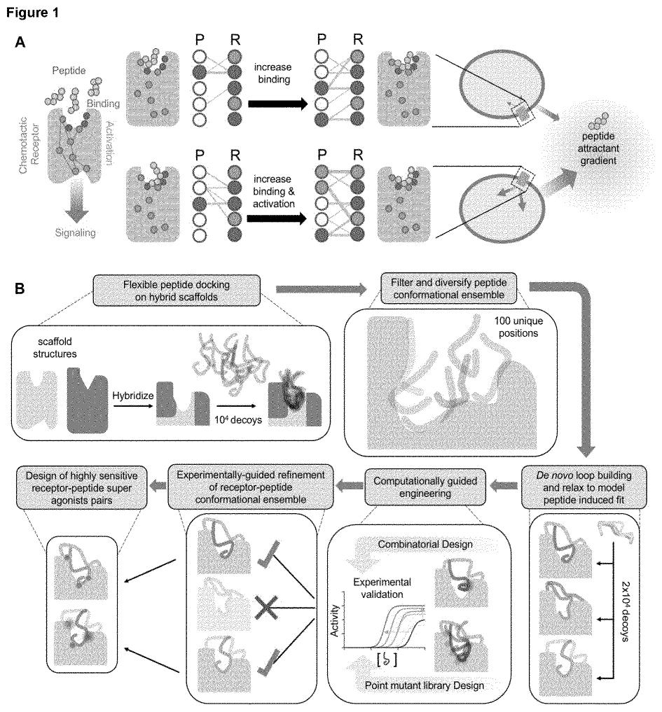

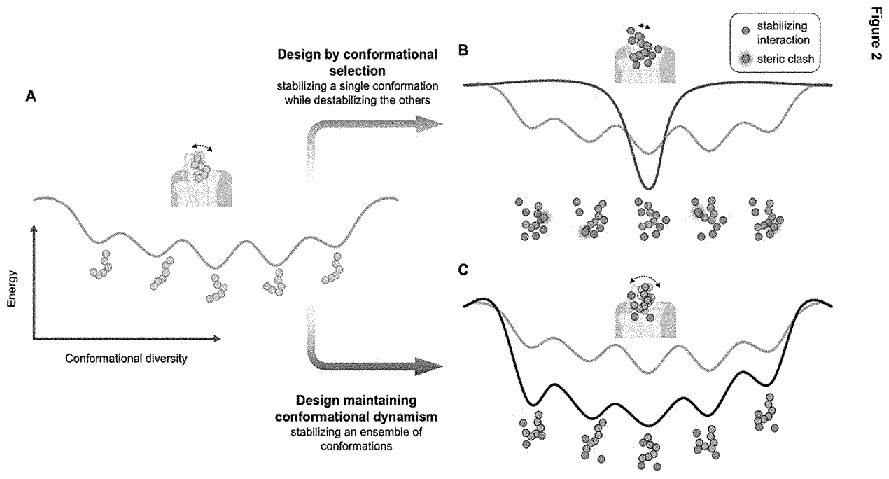

The protein scaffold decoys are preferably not modelled on the basis of the entire protein but based on those parts of the protein that participate in forming the molecular complex with the cognate peptide. For modelling the parts of the protein that participate in forming the molecular complex with the protein may be used (i) in active state conformation (i.e. with the bound cognate peptide, “ligand-bound”), or (ii) in inactive state conformation (i.e. without the bound cognate peptide, “ligand-free”), or (iii) preferably in both of these conformations. The active and inactive conformations may also be modelled based on the closest structurally characterized homology being available. In case the protein is a transmembrane receptor and the cognate peptide binds on the extracellular side only the extracellular part of the transmembrane receptor may be used. This applies mutatis mutandis to the cognate peptides. As mentioned, when investigating molecular complex interactions, it is common practice to isolate the binding motif of the disordered region and to analyze the binding as a protein-peptide interaction. Hence, the peptide decoys are generally modelled based on the part of a natural protein or peptide that is responsible for the binding in the complex. In the appended examples the decoys were modelled based on the parts of CXCL4 and CXCL12 that are shown in FIG. 1A.

After the in silico preparation of the libraries of test peptides and/or test protein scaffolds are obtained (i) the library of test peptides are docketed in silico onto the library of protein scaffolds by modelling peptide-protein molecular complexes, or (ii) the base peptide is docketed in silico on the library of protein scaffolds, or (iii) the library of test peptides is docketed in silico onto the protein or the parts of the protein that participate in forming the molecular complex with the cognate peptide.

In this connection it is to be understood that the in silico docketing of the library of test peptides onto the library of protein scaffolds results in the highest number of peptide-protein molecular complexes that can be modelled among options (i) to (iii). Option (i) is therefore preferred. It is also possible to first model the peptide-protein molecular complexes for the base peptide and the library of protein scaffolds, and/or the library of test peptides onto the protein or the parts of the protein that participate in forming the molecular complex with the cognate peptide and then select a sub-library or even only the best test peptide and/or protein scaffold for modelling the peptide-protein molecular of test peptide(s) and protein scaffold(s). The alternative options (ii) and (iii) require less peptide-protein molecular complexes that are to be modelled. For instance, in the appended examples first the lowest energy cluster was selected from the 1000 decoys of protein scaffolds and then used for further modelling with the 10000 decoys of peptides.

Non-limiting examples of protein-peptide docking programs are Rosetta FlexPepDock, HADDOCK, Pep-SiteFinder, PepCrawler, GalaxyPepDock, MDockPeP and CABS-dock; see, for example, Ghazaleh et al. (2018), Bioinformatics, 34(3):477-484, Hashemi et al. (2021), Front. Mol. Biosci., 8: 669431 or Engel et al. (2021), Synthetic and Systems Biotechnology, 6(4):402-413.

After the in silico docketing step the test peptides in the library and/or the protein scaffolds in the library are identified for which low interface energy peptide-protein molecular complexes can be modelled, preferably by a combination of flexible peptide docking and/or de novo protein structure building.

As discussed, if low interface energy peptide-protein molecular complexes can be modelled in silico it can be expected that the corresponding peptide-protein molecular complexes if generated in vitro or in vivo are stable. The low interface energy peptide-protein molecular complexes are with increasing preference the 20% or less, 10 or less and 5% or less peptide-protein molecular complexes with the lowest modelled interface energy.

The identification preferably comprises flexible peptide docking and/or de novo protein structure building.

Flexible peptide docking features significant conformational flexibility of both the peptide and the protein scaffolds during search for a binding site; see, for example, Kurcinski et al. (2019), TOOLS FOR PROTEIN SCIENCE, DOI: 10.1002/pro.3771. In computational biology, de novo protein structure prediction refers to an algorithmic process by which protein tertiary structure is predicted from its amino acid primary sequence. Algorithms and methods for de novo protein structure prediction are available, for example, from Rigden, “From Protein Structure to Function with Bioinformatics” Springer Science, 2009, ISBN 978-1-4020-9057-8.

In the example herein below Flexpepdock is used. FlexPepDock is a high-resolution peptide-protein docking (refinement) protocol for the modeling of peptide-protein complexes, implemented in the Rosetta framework. The Rosetta FlexPepDock protocol for high-resolution docking of flexible peptides mainly consists of two alternating modules that optimize the peptide backbone and rigid body orientation, respectively, using the Monte-Carlo with Minimization approach. The starting structure is refined in 200 independent FlexPepDock simulations. 100 of the simulations are carried out strictly in high-resolution mode, while 100 of the simulations include a low-resolution pre-optimization step, followed by the high-resolution refinement. A total of 200 models are thus created and then ranked based on their Rosetta generic full-atom energy score. For more details, reference is made to the method section of Raveh et al., (2010), Proteins, https://doi.org/10.1002/prot.22716.

As the next step the method of the invention comprises identifying the interacting amino acids of the test peptides and/or the protein scaffolds in the ensemble of low interface energy modelled peptide-protein molecular complexes of step (c). As discussed, this ensemble may comprise with increasing preference the 20% or less, 10 or less and 5% or less peptide-protein molecular complexes with the lowest modelled interface energy.

In this connection it is of note that ensemble modelling may generally be described as a process where multiple diverse models are created to predict an outcome, either by using many different modelling algorithms or using different training data sets. Ensemble modelling is implemented into the claimed method to mimic the dynamic nature of the receptor-peptide complex and model the diverse conformations that a peptide can adopt when binding the surface of the receptor.

After the interacting amino acids of the test peptides and/or the protein scaffolds are identified one or more of the interacting amino acids of the peptides and/or the protein scaffolds are selected and substituted in silico. The one or more interacting amino acids are with increasing preference two or more, three or more, four or more and five or more amino acids. Thereby an ensemble of peptides with substituted amino acid(s) and/or protein scaffolds with substituted amino acid(s) is obtained.

This ensemble of peptides with substituted amino acid(s) and/or the protein scaffolds with substituted amino acid(s) is again modelled into peptide-protein molecular complexes and the peptides and/or protein scaffolds for which the lowest interface energies can be modelled, correspond to engineered peptides and/or proteins being capable of forming a molecular complex that can be obtained as the final in silico product of the method of the invention. For instance, in the examples herein below four final models were selected for the designed pairs.

The desired lowest interface energy models may optionally be further validated and confirmed in silico by Principal component analysis (PCA) and/or molecular dynamics (MD).

Principal component analysis (PCA) is a process of computing principal components and using them to perform a change of basis on the data, sometimes using only the first few principal components and ignoring the rest. Principal components are in turn a collection of points in a real coordinate space and are a sequence of unit vectors, where the vector is the direction of a line that best fits the data while being orthogonal to the first i-1 vectors. Here, a best-fitting line is defined as one that minimizes the average squared distance from the points to the line. These directions constitute an orthonormal basis in which different individual dimensions of the data are linearly uncorrelated. PCA is often used in exploratory data analysis and for making predictive models.

Molecular dynamics (MD) is a computer simulation method for analyzing the physical movements of atoms and molecules. The atoms and molecules are allowed to interact for a fixed period of time, giving a view of the dynamic “evolution” of the system. In the most common version, the trajectories of atoms and molecules are determined by numerically solving Newton's equations of motion for a system of interacting particles, where forces between the particles and their potential energies are often calculated using interatomic potentials or molecular mechanics force fields. The method is applied mostly in chemical physics, materials science, and biophysics.

As can be taken from the appended examples the computational strategy according to the first aspect of the invention for engineering the interaction between a protein and a cognate peptide that are capable of forming a molecular complex results in engineered proteins and peptides that display when being tested in vitro or in vivo—as predicted by the in silico approach—high binding sensitivity and when being complexed in potent biological activity as illustrated by allosteric signal transduction responses across the membrane. Unlike previous work that only optimized binding and model receptors as rigid target structures⁹, fully flexible receptor:peptide conformational ensembles are built herein that enable the precise modeling of signaling active states and the design of complexes with novel contact networks enhancing both binding sensitivity and allosteric response (FIG. 1A). Through this approach, for example, custom-built modular biosensors can be designed that can link binding of a flexible peptide input signal to selective, fine-tuned and complex cellular responses through genetically encoded single-receptor domains. This new class of biosensors as is also called CaPSens, which stands for Conformationally-adaptive Peptide BioSensor.

In this respect it is emphasized that it is an important technical advantage of the computational strategy according to the first aspect of the invention that fully flexible receptor:peptide conformational ensembles are modelled: Hence, a plurality of fully flexible receptor:peptide conformations can be tested in parallel. On the other hand, the method used in Badaczewska-Dawid et al. (2021), Briefings in Bioinformatics, 22(3):1-9 is limited in the allowable receptor flexibility, which prohibits structural transitions to active states of the complex. Only 2 of the 7 benchmarked proteins are in a fully active ternary complex, and several models are antagonist-bound complexes. This shows that the method used in Badaczewska-Dawid et al. (2021), loc. lit. is selecting for high-affinity complexes without regard for modeling an ensemble of active receptor:peptide binding states. On the peptide side, some peptides are cyclic or have internal disulfide bonds which limit conformational flexibility, thereby limiting the conformational search space. The method in Badaczewska-Dawid et al. (2021), loc. lit. also fails to cover a wide conformational landscape of complexes. Instead, it selects for scoring features only, while the structural clustering of coarse-grained peptide docks disregards the geometric diversifying features incorporated by our algorithm. Furthermore, the method does not dock peptides in the presence of side-chain chemistry that can be important for forming key activating contacts between the peptide and the receptor. Without a proper relaxation step around a diversified set of peptide poses, the modeled induced fit effects are limited to small side-chain movements introduced by PD2 rebuilding of full-atom peptide-receptor complexes and limited side-chain movements allowed by FlexPepDock refinement.

Similarly, the modelling being involved in the method employed by Guntas et al. (2010), Proceedings of the National Academy of Science, 107(45):19256-19301 to generate a rationally selected library is very limited in conformational flexibility. There are no induced fit effects from mutual relaxation of the protein-protein interface and no remodeling of protein backbones that may be introduced by the designed sequences. There is also no incorporation of specialized modeling of peptide or loop flexibility. In designing a static interface, the strategy of Guntas et al. (2010), loc. lit. does not incorporate the conformational diversity considerations as described herein.

The computational strategy according to the first aspect of the invention is to the best knowledge of the inventors also the first approach that enables the computational design of peptide binding receptors with highly optimized binding and allosteric signaling functions. Most prior art biosensor design approaches have focused on engineering protein domains for optimal recognition of structurally well-defined molecules. By targeting flexible and structurally uncharacterized peptides, the design platform as provided herein significantly expands the range of molecules that can be detected by biosensors. Unlike approaches that rely on multi-domain sensor reconstitution upon ligand sensing, the method according to the first aspect of the invention optimizes the coupling between molecular recognition and allosteric response in a single protein domain and can generate CaPSens with unprecedented dynamic and sensitive responses. Carving biosensors into versatile GPCR (G protein-coupled receptor) scaffolds offers key additional advantages. As such, the approach provided herein paves the road for a wide range of synthetic biology, diagnostics and therapeutic applications that would benefit from sensor systems that trigger complex cellular outputs or enable direct highly sensitive detection of chemical cues.

In accordance with a preferred embodiment the protein is a receptor or a part of the receptor that is capable of binding to a natural ligand of the receptor and the cognate peptide comprises the site of the natural ligand that binds to the receptor.

In accordance with a more preferred embodiment the receptor is a G protein-coupled receptor and the natural ligand of the receptor is a peptide, preferably a chemokine.

Receptor-ligand complexes can be found in almost any cellular process. Binding of a ligand causes a conformational change in the receptor and often also in the ligand. This change generally initiates a sequence of events leading to different cellular functions.

The receptor is preferably a G protein-coupled receptor (GPCR), also known as seven-(pass)-transmembrane domain receptor, 7-TM receptor, heptahelical receptor, serpentine receptor, or G protein-linked receptor (GPLR). Such receptors form a large group of evolutionarily-related proteins that are cell surface receptors that detect molecules outside the cell and activate cellular responses. Coupling with G proteins, they are called seven-transmembrane receptors because they pass through the cell membrane seven times. Ligands can bind either to extracellular N-terminus and loops (e.g. glutamate receptors) or to the binding site within transmembrane helices (Rhodopsin-like family). They are all activated by agonists.

GPCRs are also an important drug target and approximately 34% of all Food and Drug Administration (FDA) approved drugs target 108 members of this family. The global sales volume for these drugs is estimated to be 180 billion US dollars as of 2018. It is estimated that GPCRs are targets for about 50% of drugs currently on the market, mainly due to their involvement in signalling pathways related to many diseases, i.e. mental, metabolic including endocrinological disorders, immunological including viral infections, cardiovascular, inflammatory, senses disorders, and cancer.

GPCRs include one or more receptors for the following ligands: sensory signal mediators (e.g., light and olfactory stimulatory molecules); adenosine, bombesin, bradykinin, endothelin, γ-aminobutyric acid (GABA), hepatocyte growth factor (HGF), melanocortins, neuropeptide Y, opioid peptides, opsins, somatostatin, GH (growth hormone), tachykinins, members of the vasoactive intestinal peptide family, and vasopressin; biogenic amines (e.g., dopamine, epinephrine, norepinephrine, histamine, serotonin, and melatonin); glutamate (metabotropic effect); glucagon; acetylcholine (muscarinic effect); chemokines; lipid mediators of inflammation (e.g., prostaglandins, prostanoids, platelet-activating factor, and leukotrienes); peptide hormones (e.g., calcitonin, C5a anaphylatoxin, follicle-stimulating hormone [FSH], gonadotropin-releasing hormone [GnRH], neurokinin, thyrotropin-releasing hormone [TRH], and oxytocin); and endocannabinoids.

GPCR structures are composed of 3 main regions: the extracellular ligand binding pocket, the intracellular G-protein binding domain and the “transmission” transmembrane (TM) region which connects the two binding regions and allows them to communicate. GPCRs typically switch from inactive to active state conformations upon activating agonist ligand and G-protein binding at the extracellular and intracellular domains, respectively. The structural rearrangements upon receptor activation involve large intracellular reorientations of transmembrane helices (TMH), notably TMH6 and TMH7 and smaller scale movements of individual amino acids across the entire TM domain.

In the appended examples the method of the first aspect of the invention is illustrated based on the protein-ligand complex of the GPCR CXC4R and its ligand CXCL12.

C-X-C chemokine receptor type 4 (CXCR-4) also known as fusin or CD184 (cluster of differentiation 184) is a protein that in humans is encoded by the CXCR4 gene. The protein is a CXC chemokine receptor.

The stromal cell-derived factor 1 (SDF1), also known as C-X-C motif chemokine 12 (CXCL12), is a chemokine that in humans is encoded by the CXCL12 gene on chromosome 10.

In accordance with a further preferred embodiment the method further comprises (g) further selecting and substituting in silico one or more of the interacting amino acids of the peptides and/or the protein scaffolds as identified in step (d) based on the ensemble of peptides with substituted amino acid(s) and/or the protein scaffolds with substituted amino acid(s) for which the lowest interface energy peptide-protein molecular complexes can be modelled of (f); and (h) generating in silico based on the peptides with substituted amino acid(s) and/or the protein scaffolds with substituted amino acid(s) of (g) an ensemble of peptides with substituted amino acid(s) and/or the protein scaffolds with substituted amino acid(s) for which the lowest interface energy peptide-protein molecular complexes can be modelled, thereby obtaining further engineered peptides and/or proteins being capable of forming a molecular complex.

Also in step (e) one or more of the interacting amino acids of the peptides and/or the protein scaffolds as identified are selecting and substituted and also in step (f) the peptides with substituted amino acid(s) and/or the protein scaffolds with substituted amino acid(s) are then used to generate an ensemble of peptides with substituted amino acid(s) and/or the protein scaffolds with substituted amino acid(s) for which the lowest interface energy peptide-protein molecular complexes can be modelled.

As is illustrated by FIG. 1 during steps (e) and (f) so-called first generation engineered peptides and/or proteins being capable of forming a molecular complex are obtained while on top of that in accordance with the above preferred embodiment and the additional steps (g) and (h) so-called second generation engineered peptides and/or proteins being capable of forming a molecular complex are obtained.

The interface energy of the peptide-protein molecular complexes of the second generation is generally lower than the interface energy of the peptide-protein molecular complexes of the first generation. It follows that the binding affinity of the second generation engineered peptides and/or proteins being capable of forming a molecular complex is generally higher than of the engineered peptides and/or proteins being capable of forming a molecular complex of the first generation.

In accordance with a preferred embodiment the method further comprises after step (d) (e′) selecting and substituting in silico selected single amino acids of the interacting amino acids of the peptides and/or the protein scaffolds as identified in (d) based on the ensemble of low interface energy models of peptide-protein molecular complexes as identified in step (c); (f′) identifying the peptides and/or protein scaffolds with single substituted amino acid for which the lowest interface energy models of peptide-protein molecular complexes can be modelled; (g′) generating in silico based on the peptides and/or the protein scaffolds as identified in step (f′) and the peptides and/or the protein scaffolds as identified in step (f) and/or (h) engineered peptides and/or proteins that each carry at least one substituted interacting amino acid position as identified in step (e′) and at least one substituted interacting amino acid position as identified in step (f) and/or (h) for which the lowest interface energy models of peptide-protein molecular complexes can be modelled.

The at least one substituted interacting amino acid position is for each occurrence independently preferably at least two, more preferably at least three and most preferably at least four substituted interacting amino acid positions.

In accordance with a further preferred embodiment the one or more interacting amino acids of step (e) and/or (g) are at least two amino acids that can be found with the same domain of the protein or a protein scaffold, preferably in a putative binding pocket of the protein or a protein scaffold.

With respect to the above two preferred embodiments it is of note that in steps (e) and (g) one or more of the interacting amino acids of the peptides and/or the protein scaffolds are selected and substituted while in step (e′) single amino acids of the interacting amino acids of the peptides and/or the protein scaffolds are substituted. The different single amino acids substituted in step (e′) can therefore further downstream be combined to yield peptides/protein scaffolds with different single amino acids substitutions.

The interacting amino acids in the protein (on the basis of which the protein scaffolds were obtained) and/or the protein scaffolds assemble a particular three-dimensional configuration for binding to a peptide. Within this three-dimensional configuration two or more interacting amino acids can often be found in the same domain of the protein or a protein scaffold, such as in a putative binding pocket of the protein or a protein scaffold. The domain or putative binding pocket may comprise continuous amino acids that can be found in the amino acid sequence of the protein or a protein scaffold next to each other or almost next to each other. The domain or putative binding pocket may also comprise discontinuous amino acids that only form a domain or putative binding pocket in the particular three-dimensional configuration for binding to a peptide. In biochemistry and molecular biology, a binding pocket is generally a region on a macromolecule such as a protein that binds to another molecule with specificity. The amino acids of such a domain, preferably putative binding site are preferably selected and substituted together in step (e) and/or (g).

On the other hand, also single interacting amino acids that cannot be found with one or more other interacting amino acids in the same domain or binding pocket may contribute to the formation of the peptide-protein molecular complexes. Such interacting amino acids may, for example, help to position the peptide into the binding domain, preferably binding pocket. Such single interacting amino acids are selected and substituted in accordance with step (e′) and in step (f′) the peptides and/or protein scaffolds with single substituted amino acid for which the lowest interface energy models of peptide-protein molecular complexes can be modelled are determined.

One or more amino acid substitutions of the first and/or second generation engineered peptides and/or proteins as identified in step (f) and/or (h) can be combined with the engineered peptides and/or proteins as identified in step (f′) in step (g′) which then finally results in yet further engineered peptides and/or proteins being capable of forming a molecular complex.

The further implementation of the in silico generation of peptides and/or proteins with single substituted amino acid into the claimed method may result in peptide-protein molecular complexes displaying an even lower modelled interface energy than the peptide-protein molecular complexes of the first and second generation.

In accordance with a preferred embodiment the method further comprises the production of (i) the engineered peptides and/or proteins as identified in step (f) or (h) or as generated in step (g′) or peptides; and/or (ii) proteins that comprise peptides and/or proteins as identified in step (f) or (h) or as generated in step (g′) by peptide and/or protein synthesis or site-directed mutagenesis.

As discussed herein above, so far all steps of the claimed method were carried out in silico and the final “product” are modelled amino acid sequences of engineered peptides and/or proteins being capable of forming a molecular complex.

In accordance with the above preferred embodiment these engineered peptides and/or proteins are actually produced by peptide and/or protein synthesis or by site-directed mutagenesis.

Means and methods for protein and peptide synthesis are known in the art; see, for example, Albericio and Govender (2012), Special Issue “Chemical Protein and Peptide Synthesis”, ISSN 1420-3049.

It is also possible to introduce selected amino acid substitutions into naturally occurring proteins and peptides (site-directed mutagenesis), so that the naturally occurring proteins and peptides are engineered to proteins and peptides being based on the naturally occurring proteins and peptides that comprise the engineered peptides and/or proteins as identified in step (f) or (h) or as generated in step (g′).

As discussed above, engineered peptides and/or proteins are often only comprised of parts of naturally occurring protein and peptides. For this reason it is also of interest to generate peptides and/or proteins that comprise peptides and/or proteins as identified in step (f) or (h) or as generated in step (g′). In this respect it is preferred that peptides and/or proteins as identified in step (f) or (h) or as generated in step (g′) are comprised in proteins and peptides that in addition comprise the parts of the naturally occurring protein and peptides on the basis of which peptides and/or proteins as identified in step (f or (h) or as generated in step (g′) were obtained and that cannot be found in the peptides and/or proteins as identified in step (f) or (h) or as generated in step (g′) per se. For instance and as discussed above, in the examples of the application the in silico-modelled peptides and proteins correspond parts of CXCL12 and CXCR4. Hence, full-length derivative of CXCL12 and CXCR4 may be produced wherein the wild-type parts CXCL12 and CXCR4 are replaced by the in silico-modelled peptides and proteins as obtained by the method of the invention.

The produced peptides may be C-terminally amidated in order to avoid unwanted charge effects of the carboxy terminus in further experiments or uses.

In accordance with a more preferred embodiment the method further comprises the step (i) validation of at least one synthesized or mutated peptide and/or protein in a functional assay, preferably a cell-based functional assay that allows to monitor the formation of a molecular complex between a protein and a peptide.

As discussed herein above, the peptides and/or proteins as identified in step (f or (h) or as generated in step (g′) were modelled so that they are capable of forming particularly strong molecular complexes and it is demonstrated in the appended examples that the engineered peptides and/or proteins even allow for the generation of superagonistic peptides.

In accordance with step (i) the predicted formation of a molecular complex between a protein and peptide is tested in a functional assay, preferably a cell-based functional assay. The functional assay, preferably a cell-based functional assay may be carried out in vitro or in vivo and is preferably carried out in vitro.

Suitable functional assays are illustrated in the appended examples and comprise in the case of an GPCRs as the base for the protein assays for example G-protein Gi activation (see section “Gai dissociation BRET” in the examples) or Ca²⁺ mobilization (see, for example, Wosczek and Fuerst (2015), Methods Mol Biol, 1272:79-89) or cell migration (see section “Migration assays” in the examples).

In accordance with a more preferred embodiment, the method further comprises (j) combining a synthesized or mutated peptide and/or protein into a molecular complex with another synthesized or mutated protein and/or peptide or the native protein or cognate peptide into a molecular complex; (k) identifying superagonistic pairs of proteins and peptides; and (I) optionally further refining the superagonistic pairs of proteins and peptides by substituting one or more of the interacting amino acids of the protein/peptide pairs and the identification of protein/peptide pairs that display and improved superagonistic activity, binding selectivity or binding orthogonality as compared to the superagonistic pairs of proteins and peptides of (k).

In accordance with this preferred embodiment the peptides and/or proteins as identified in step (f) or (h) or as generated in step (g′) are allowed to form molecular complexes, generally in vitro. Then superagonistic pairs of proteins and peptides are identified by suitable test, such as a cell migration assay. Once the superagonistic pairs are identified, they may optionally be further refined in accordance with step (l).

In the field of pharmacology, a superagonist is a type of agonist that is capable of producing a maximal response greater than the endogenous agonist for the target receptor, and thus has an efficacy of more than 100% as compared to the endogenous agonist. It is demonstrated in the examples that the superagonistic pairs of proteins and peptides that were generated by the claimed method based on CXCR4 and CXCL12 are ultrasensitive chemotactic pairs eliciting potent chemotaxis in human primary T-cells as the final result of the modelled enhanced contacts. An unprecedented signalling efficacy and potency was achieved. The superagonistic pairs of proteins and peptides have the potential to mature into therapeutic applications.

The present invention relates in a second aspect to a variant of a human CXCR4-derived protein (A) as characterized by an amino acid sequence comprising or consisting of SEQ ID NO: 1, wherein at least two, preferably at least three of (i) to (viii) apply: (i) amino acid position 37 is any other amino acid than N and is preferably A, (ii) amino acid position 41 is any other amino acid than L and is preferably A or I, and is most preferably I, (iii) amino acid position 45 is any other amino acid than Y and is preferably F, (iv) amino acid position 113 is any other amino acid than H and is preferably A, M, or N, and is most preferably N, (v) amino acid position 178 is any other amino acid than S and is preferably F or A and is most preferably A, (vi) amino acid position 181 is any other amino acid than D and is preferably Q, (vii) amino acid position 185 is any other amino acid than I and is preferably V, and (viii) amino acid position 285 is any other amino acid than S and is preferably M, (B) sharing at least 80% sequence identity with the CXCR4-derived protein of (A), provided that at least two, preferably at least three of (i) to (viii) as defined in (A) apply, (C) being selected from amino acid sequences comprising or consisting of SEQ ID NOs 3 to 6, or (D) according to one of (A) to (C), wherein the signal peptide is absent.

SEQ ID NO: 1 is wild-type human CXCR4. SEQ ID NO: 2 is wild-type human CXCR4 with a 3×HA-tag at the N-terminus. SEQ ID Nos 3 to 6 corresponds to the best variants of a human CXCR4-derived proteins as obtained in the appended examples. The at least two, preferably at least three of (i) to (viii) are with increasing preference at least four of (i) to (viii) and at least five of (i) to (viii).

The present invention relates in a third aspect to a variant of a human CXCL12-derived peptide (A) as characterized by an amino acids sequence comprising or consisting of SEQ ID NO: 7, wherein (i) amino acid position 3 is any other amino acid than V and is preferably L, F, W, or Y, and is most preferably Y, and/or (ii) amino acid position 7 is any other amino acid than V and is preferably L, (B) sharing at least 80% sequence identity with the CXCR4-derived protein of (A), provided that (i) and/or (ii) as defined in (A) apply, or (C) as selected from an amino acid sequence comprising or consisting of SEQ ID NOs 8 to 10.

SEQ ID NO: 7 is wild-type human CXCL12. SEQ ID Nos 8 to 10 corresponds to the best variants of a human CXCL12-derived peptides as obtained in the appended examples. The sequence share of at least 80% identity is with increasing preference at least 85%, at least 90%, at least 95%, at least 96%, at least 97%, at least 98%, and at least 99% identity. On the other hand, it is also described herein that sequence share of at least 80% identity may also only be at least 70% identity, be at least 65% identity or at least 60% identity.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment, for two or more sequences or subsequences the percentage of amino acid residues or nucleotides that are the same (e.g. 70%, 75%, 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences. The NCBI BLAST algorithm is available for protein (Protein BLAST) and nucleotides (Nucleotide BLAST). For Protein BLAST the algorithm parameters are preferably: max target sequences: 100, with automatically adjust parameters for short input sequences, expect threshold 0.05, word size 6, Max matches in a query range 0, matrix BLOSUM62, cap cost existence: 10 extension: 1, and compositional adjustment. For Nucleotide BLAST the algorithm parameters are preferably: max target sequences: 100, with automatically adjust parameters for short input sequences, Expect threshold 0.05, word size 28, Max matches in a query range 0, match/mismatch scores 1,-2, cap costs linear, low complexity regions filter, and, ask for look up table only. These are the standard algorithm parameters for protein BLAST and Nucleotide BLAST and they can adjusted, if needed.

As discussed herein above, the claimed method is illustrated in the appended examples based on the molecular complex that is formed in nature by CXCR4 and CXCL12. The engineered peptides and/or proteins as obtained in the examples based CXCR4 and CXCL12 form the basis of the variant of a human CXCR4-derived protein and the variant of a human CXCL12-derived peptide of the second and third aspect of the invention.

The amino acids as listed in the second and third aspect of the invention correspond to selected interacting amino acids that were substituted in silico and later in vitro and for which improved binding sensitivity and/or specificity was obtained upon their substitutions.

Hence, the substitution of the naturally occurring amino acids at these positions in CXCR4 and CXCL12 allows to generate variants of a CXCR4-derived protein and a CXCL12-derived peptide displaying an altered binding specificity and/or sensitivity. In particular, the listed preferred and most preferred amino acids that are to replace the corresponding naturally amino acids were found to result in variant displaying improved specificity and/or sensitivity.

According to a preferred embodiment, the variant of a CXCR4-derived protein and/or a CXCL12-derived peptide is a fusion protein.

A “fusion protein” according to the present invention contains at least one additional heterologous amino acid sequence other than the variant of a CXCR4-derived protein and/or a CXCL12-derived peptide. Often, but not necessarily, these additional sequences will be located at the N- or C-terminal end of the (poly)peptide. It may e.g. be convenient to initially express the (poly)peptide as a fusion protein from which the additional amino acid residues can be removed, e.g. by a proteinase capable of specifically trimming the fusion protein and releasing the (poly)peptide of the present invention. The additional heterologous amino acid sequence can either be directly or indirectly fused to the variant of a CXCR4-derived protein and/or a CXCL12-derived peptide of the invention. In case of an indirect fusion generally a peptide linker may be used for the fusion, such as a GS-linker.

Those at least one additional heterologous amino acid sequence of said fusion proteins includes amino acid sequences which confer desired properties such as modified/enhanced stability, modified/enhanced solubility and/or the ability of targeting one or more specific cell types. For example, fusion proteins with antibodies are envisioned herein. The term “antibody” comprises antibody fragments and derivatives. The antibody may be, for example, specific for cell surface markers or may be an antigen-recognizing fragment of said antibodies. The protein or peptide of the invention can be fused to the N-terminus or C-terminus of the light and/or heavy chain(s) of an antibody. The protein or peptide of the invention is preferably fused to the N-terminus of the light and/or heavy chain(s) of an antibody, so that the Fc part of the antibody is free to bind to Fc-receptors.

The term “antibody” as used in accordance with the present invention comprises, for example, polyclonal or monoclonal antibodies. Furthermore, also derivatives or fragments thereof, which still retain the binding specificity to the desired target, e.g. a tumor antigen, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler EP, Serebryanaya DV, Katrukha AG. 2010, Biochemistry (Mosc)., vol. 75(13), 1584; Holliger P, Hudson PJ. 2005, Nat Biotechnol., vol. 23(9), 1126). The multimeric formats in particular comprise bispecific antibodies that can simultaneously bind to two different types of antigen. The first antigen can be found on the protein of the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

The fusion protein may also comprise protein domains known to function in signal transduction and/or known to be involved in protein-protein interaction. Examples for such domains are Ankyrin repeats; arm, Bcl-homology, Bromo, CARD, CH, Chr, C1, C2, DD, DED, DH, EFh, ENTH, F-box, FHA, FYVE, GEL, GYF, hect, LIM, MH2, PDZ, PB1, PH, PTB, PX, RGS, RING, SAM, SC, SH2, SH3, SOCS, START, TIR, TPR, TRAF, tsnare, Tubby, UBA, VHS, W, WW, and 14-3-3 domains. Further information about these and other protein domains is available from the databases InterPro (http://www.ebi.ac.uk/interpro/, Mulder et al., 2003, Nucl. Acids. Res. 31: 315-318), Pfam (http://www.sanger.ac.uk/Software/Pfam/, Bateman et al., 2002, Nucleic Acids Research 30(1): 276-280) and SMART (http://smart.embl-heidelberg.de/, Letunic et al., 2002, Nucleic Acids Res. 30(1), 242-244).

The at least one additional heterologous amino acid sequence of the fusion protein according to the present invention may comprise or consist of (a) a cytokine, (b) a chemokine, (c) a pro-coagulant factor, (d) a proteinaceous toxic compound, and/or (e) an enzyme for pro-drug activation.

The cytokine is preferably selected from the group consisting of IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1alpha and IL-1 beta. As it is well-known in the art, cytokines may favour a pro-inflammatory or an anti-inflammatory response of the immune system. Thus, depending on the disease to be treated either fusion proteins with a pro-inflammatory or an anti-inflammatory cytokine may be favored. For example, for the treatment of inflammatory diseases in general fusion constructs comprising anti-inflammatory cytokines are preferred, whereas for the treatment of cancer in general fusion constructs comprising pro-inflammatory cytokines are preferred.

The chemokine is preferably selected from the group consisting of IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine. The major role of chemokines is to act as a chemoattractant to guide the migration of cells. Cells that are attracted by chemokines follow a signal of increasing chemokine concentration towards the source of the chemokine. It follows that within the fusion protein the chemokine can be used to guide the migration of the protein or peptide of the invention, e.g. to a specific cell type or body site.

The pro-coagulant factor is preferably a tissue factor. A pro-coagulant factor promotes the process by which blood changes from a liquid to a gel, forming a blood clot. Pro-coagulant factors may, for example, aid in wound healing.

The proteinaceous toxic compound is preferably Ricin-A chain, modeccin, truncated Pseudomonas exotoxin A, diphtheria toxin or recombinant gelonin. Toxic compounds can have a toxic effect on a whole organism as well as on a substructure of the organism, such as a particular cell type. Toxic compounds are frequently used in the treatment of tumors. Tumor cells generally grow faster than normal body cells, so that they preferentially accumulate toxic compounds and in higher amounts.

The enzyme for pro-drug activation is preferably an enzyme selected from the group consisting of carboxy-peptidases, glucuronidases and glucosidases. Among the broad array of genes that have been evaluated for tumor therapy, those encoding pro-drug activation enzymes are especially appealing as they directly complement ongoing clinical chemotherapeutic regimes. These enzymes can activate prodrugs that have low inherent toxicity using both bacterial and yeast enzymes or enhance prodrug activation by mammalian enzymes.

The fusion protein may also comprise a tag, such as purification tag. Several purification tags are available and an overview of affinity tags for protein purification is available in Kimple et al. (2013), Curr Protoc Protein Sci. 2013; 73: Unit-9.9. In the examples a HA tag (3×HA) is illustrated.

In accordance with a preferred embodiment, the variant of a CXCR4-derived protein and/or a CXCL12-derived peptide is fused to a heterologous non-proteinaceous compound.

As used herein, a heterologous compound is a compound that cannot be found in nature fused to CXCR4 and CXCL12.

The heterologous non-proteinaceous compound can either be directly or indirectly fused to the variant of a CXCR4-derived protein and/or a CXCL12-derived peptide. For example, a chemical linker may be used. Chemical linkers may contain diverse functional groups, such as primary amines, sulfhydryls, acids, alcohols and bromides. Many crosslinkers are functionalized with maleimide (sulfhydral reactive) and succinimidyl ester (NHS) or isothiocyanate (ITC) groups that react with amines.

The heterologous non-proteinaceous compound is preferably a pharmaceutically active compound or diagnostically active compound. The pharmaceutically active compound or diagnostically active compound is preferably selected from the group consisting of (a) a fluorescent dye, (b) a photosensitizer, (c) a radionuclide, (d) a contrast agent for medical imaging, (e) a toxic compound, or (f) an ACE inhibitor, a Renin inhibitor, an ADH inhibitor, an Aldosteron inhibitor, an Angiotensin receptor blocker, a TSH-receptor, a LH-/HCG-receptor, an oestrogen receptor, a progesterone receptor, an androgen receptor, a GnRH-receptor, a GH (growth hormone) receptor, or a receptor for IGF-I or IGF-II.

The fluorescent dye is preferably a component selected from Alexa Fluor or Cy dyes.

The photosensitizer is preferably phototoxic red fluorescent protein KillerRed or haematoporphyrin.

The radionuclide is preferably either selected from the group of gamma-emitting isotopes, more preferably ^{99m}Tc, ¹²³I, ¹¹¹In, and/or from the group of positron emitters, more preferably ¹⁸F, ⁶⁴Cu, ⁶⁸Ga, ⁸⁶Y, ¹²⁴I and/or from the group of beta-emitters, more preferably ¹³¹I, ⁹⁰Y, ¹⁷⁷Lu, ⁶⁷CU, ⁹⁰Sr, and/or from the group of alpha-emitters, preferably ²¹³Bi, ²¹¹At.

A contrast agent as used herein is a substance used to enhance the contrast of structures or fluids within the body in medical imaging. Common contrast agents work based on X-ray attenuation and magnetic resonance signal enhancement.

The toxic compound is preferably a small organic compound, more preferably a toxic compound selected from the group consisting of calicheamicin, maytansinoid, neocarzinostatin, esperamicin, dynemicin, kedarcidin, maduropeptin, doxorubicin, daunorubicin, and auristatin. In contrast to the herein above described proteinaceous toxic compound these toxic compounds are non-proteinaceous.

The present invention relates in a fourth aspect to a nucleic acid molecule, preferably a vector encoding the variant of the human CXCR4-derived protein of the invention and/or the variant of the human CXCL12-derived peptide of the invention.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may additionally comprise promoters, enhancers, response elements, signal sequences, polyadenylation sequences, introns, 5′- and 3-non-coding regions, and the like.

The term “vector” in accordance with the invention means preferably a plasmid, cosmid, virus, bacteriophage or another vector used e.g. conventionally in genetic engineering which carries the nucleic acid molecule of the invention. The nucleic acid molecule of the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically-designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The vector is preferably a retroviral vector. A retroviral vector consists of proviral sequences that can accommodate the gene of interest, to allow incorporation of both into the target cells. In the appended examples T-cells are transduced with retroviral vectors encoding the variant of the human CXCR4-derived protein of the invention.

The present invention relates in a fifth aspect to a cell, preferably a lymphocyte and most preferably a T-cell comprising the nucleic acid molecule, preferably the vector of the invention.

The cell is preferably an in vitro cell and not an in vivo cell. The cell may also be referred to as a “host cell”.

The term “host cell” means any cell of any organism that is selected, modified, transformed, grown, or used or manipulated in any way, for the production of the variant of the CXCR4-derived protein of the invention and/or the variant of the human CXCL12-derived peptide of the invention by the cell.

The host cell of the invention is typically produced by introducing the nucleic acid molecule or vector(s) of the invention into the host cell which upon its/their presence mediates the expression of the nucleic acid molecule of the invention encoding the CXCR4-derived protein of the invention and/or the variant of the human CXCL12-derived peptide and/or the fusion proteins of the invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli (e.g., E coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may a Saccharomyces cerevisiae cell, Pichia pastoris cell or an Aspergillus cell. Preferred examples for host cell to be genetically engineered with the nucleic acid molecule or the vector(s) of the invention is a cell of yeast, E. coli and/or a species of the genus Bacillus (e.g., B. subtilis). In one preferred embodiment the host cell is a yeast cell (e.g. S. cerevisiae).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucell's Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

A lymphocyte is a type of white blood cell in the immune system of jawed vertebrates. Lymphocytes include, innate lymphoid cells (ILCs, i.e. innate counterparts of T cells that contribute to immune responses by secreting effector cytokines and regulating the functions of other innate and adaptive immune cells), natural killer cells (which function in cell-mediated, cytotoxic innate immunity), T cells (for cell-mediated, cytotoxic adaptive immunity), and B cells (for humoral, antibody-driven adaptive immunity).

The lymphocyte is preferably an anti-tumor lymphocyte. An anti-tumor lymphocyte (or an anti-tumor effector lymphocyte) is a lymphocyte capable of eliciting a cytolytic response that can cause tumor cell death. These lymphocytes are specializing in and equipped for tumor cell elimination. The first category encompasses clonally expanded T lymphocytes expressing a unique T cell receptor (TCR) and recognizing tumor epitopes in the context of the major histocompatibility complex (MHC) molecules. These T cells, optionally together with B cells producing tumor-specific antibodies and dendritic cells (DC) processing and presenting tumor epitopes, can mediate an adaptive immunity against tumors. The second category of effector cells includes natural killer (NK) cells, NK-T cells, and macrophages (M). These cells are not restricted by the MHC molecules in their interactions with tumor targets, and they mediate innate immunity. Each type of effector cells, whether specific or nonspecific, contains subsets of cells at different stages of differentiation and activation. This means that each type of effector potentially able to target tumor cells contains a heterogeneous mix of cells with distinct functional capabilities, depending on their stage of differentiation, maturation, and/or activation (Holland, Frei; Cancer Medicine; 6th edition. chapter “Antitumor Effector Cells in Humans”). All these types of anti-tumor lymphocytes are envisioned in accordance with the present invention.

The lymphocytes are preferably T-cells or NK cells, whereby T-cells are further preferred.

A T-cell or T-lymphocyte can be distinguished from other lymphocytes by the presence of a T-cell receptor (TCR) on its cell surface. One of the functions of T-cells is mediating immune-mediated cell death, and it is carried out by two major subtypes: CD8+“killer” and CD4+“helper” T-cells. CD8+ T cells are cytotoxic which means that they are able to directly kill selected cell. These selected cells are in accordance with the invention tumor cells, virus-infected cells, as well as cancer cells. CD4+ cells function as “helper cells”. Unlike CD8+ killer T-cells, these CD4+ helper T-cells function by further activating memory B cells and cytotoxic T-cells, which leads to a larger immune response which is in accordance with the invention directed against tumor cells. The specific adaptive immune response regulated by the T-helper cell depends on its subtype, which is distinguished by the types of cytokines they secrete. The T-cells are preferably a CD8+ killer T-cells or mixture of CD8+ killer and CD4+ helper T-cells.

A natural killer (NK) cell is a type of cytotoxic lymphocyte being critical to the innate immune system that belong to the rapidly expanding family of innate lymphoid cells (ILC) and represent 5-20% of all circulating lymphocytes in humans. The role of NK cells in innate immune system is analogous to that of cytotoxic T-cells in the vertebrate adaptive immune response. NK cells provide rapid responses to virus-infected cells and other intracellular pathogens acting at around 3 days after infection and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because they do not require activation to kill cells that are missing “self” markers of MHC class 1. This role is especially important because harmful cells that are missing MHC I markers cannot be detected and destroyed by other immune cells, such as T-cells.

The lymphocytes may also be chimeric antigen receptor T-cells (CAR T-cells), T-cell-receptor-engineered T-cells (TCR T-cells), chimeric antigen receptor NK-cells (CAR NK-cells), NK cell receptor-engineered NK cells (NCR NK-cells), TCR/CAR hybrid T-cells, NCR/CAR hybrid NK-cells or tumor-infiltrating lymphocytes (TILs).

Chimeric antigen receptor T-cells (CAR) T-cells are T-cells that have been genetically engineered to produce a chimeric T cell receptor (CAR) for use in immunotherapy. The receptors are chimeric because they combine both antigen-binding and T-cell activating functions into a single receptor. CAR-T cell therapy uses T-cells engineered with CARs for cancer therapy. The premise of CAR-T immunotherapy is to modify T-cells to recognize tumor cells in order to more effectively target and destroy them in order to generate CAR T-cells. T-cells are harvested from subject, genetically altered, and then infused into patients to attack a tumor in a subject. CAR T-cells can be both CD4+ and/or CD8+ cells. A 1-to-1 ratio of both cell types is preferred since it provides synergistic antitumor effects.

CAR T-cells are engineered to transfer arbitrary specificity onto an immune effector cell, like a T cell, which specifically eliminates antigen-bearing tumor cells. The CAR may comprise a scFv being derived from an antibody, a CDζ (and a transmembrane domain (so-called first-generation CARs). In this way, the engineered CAR is able to recognize specific tumor associated-antigens. Therefore, the CAR has the ability to bind unprocessed tumor surface antigens without MHC processing while TCRs engage with both tumor intracellular and surface antigenic peptides embedded in MHC.

In contrast, TCRs are α/β heterodimers that bind to the MHC-bound antigens. As discussed above, CARs recognize tumor antigen which led to T-cell activation with different functions compared with TCRs. CAR-T cell therapy has certain disadvantages like off-tumor toxicities when targeting tumor-specific antigen. Compared with CARs, TCRs have several structural advantages in T cell-based therapy, such as more subunits in their receptor structure (ten subunits vs one subunit), greater immunoreceptor tyrosine-based activation motif (ITAMs) (ten vs three), less dependence on antigens (one vs 100), and more co-stimulate receptors (CD3, CD4, CD28, etc.) (Zhao et al. (2021) Front. Immunol., |https://doi.org/10.3389/fimmu.2021.658753).

CAR NK-cells are distinguished from CAR T-cells in that the chimeric antigen receptor is introduced into NK cells instead of T-cells. Just as CAR T-cells, CAR-NK cells can be engineered to target diverse antigens, enhance proliferation and persistence in vivo, increase infiltration into solid tumours, overcome resistant tumour microenvironment, and ultimately achieve an effective anti-tumour response.

Natural cytotoxicity receptor NK cells (NCR NK-cells) are NK-cell that have been genetically engineered to express a NCR. The NCRs have been proposed to bind to many cellular ligands which are implicated in NK cell surveillance of tumor cells. Many of these interactions have been shown to evoke the cytotoxic and cytokine-secreting functions of NK cells. However, it is also possible that the NCRs may regulate other anti-tumor pathways. NCRs and their ligands can be successfully targeted for cancer immunotherapy. NCRs have been classically defined as activating receptors delivering potent signals to NK cells in order to lyse harmful cells and to produce inflammatory cytokines.

TCR/CAR hybrid T-cells are T-cells that have been genetically engineered to express a TCR and CAR. Similarly, NCR/CAR hybrid NK-cells are T-cells that have been genetically engineered to express a NCR and CAR.

Tumor-infiltrating lymphocytes (TILs) are white blood cells that have left the bloodstream and migrated towards a tumor. TILs are implicated in killing tumor cells. The presence of lymphocytes in tumors is often associated with better clinical outcomes.

In accordance with a more preferred embodiment of the invention, the tumor-infiltrating lymphocytes are tumor-infiltrating T-cells or tumor-infiltrating NK cells.

In adoptive T-cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. TILs are typically expanded over the course of a few weeks with a high dose of IL-2 in 24-well plates. Selected TIL lines that presented best tumor reactivity are then further expanded in a “rapid expansion protocol” (REP), which uses anti-CD3 activation for a typical period of two weeks. The final post-REP TIL is infused back into a patient in order to treat a tumor of the patient. This applies mutatis mutandis to adoptive NK-cell transfer with TILs.

In accordance with a further preferred embodiment of the invention the anti-tumor lymphocytes are autologous anti-tumor lymphocytes.

In an anti-tumor therapy with autologous lymphocytes the lymphocytes are taken from a subject having a tumor and are genetically engineered (e.g. to produce CAR T-cells) and/or selected and/or expanded (e.g. to produce TILs) ex vivo and then transferred back into the same subject. These autologous therapies are subject-specific because the therapeutic cells are created from a subject's own cells.

The present invention relates in a sixth aspect to a molecular complex comprising the variant of the human CXCR4-derived protein of the invention and/or the variant of the human CXCL12-derived peptide of the invention.

As discussed herein above, the variant of the human CXCR4-derived protein of the invention and the variant of the human CXCL12-derived peptide of the invention are capable of forming a molecular complex with each other. The variant of human CXCR4-derived protein of the invention can form a complex with human CXCL12 and other natural ligands of the CXCR4. Similarly, the variant of the human CXCL12-derived peptide of the invention can form a complex with human CXCR4 and other naturally occurring receptors, in particular other CXC-receptors. Such molecular complexes are the subject of the sixth aspect of the present invention. The molecular complexes may be formed, for example, within a cell and also within in a suitable medium.

The present invention relates in a seventh aspect to a composition, preferably a diagnostic or pharmaceutical composition, or a kit comprising the variant of a human CXCR4-derived protein of the invention, the variant of a human CXCL12-derived peptide, the nucleic acid molecule, the vector, the host cell of the invention or a combination thereof.

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. The length of treatment needed to observe changes and the interval following treatment for responses to occur vary depending on the desired effect. The particular amounts may be determined by conventional tests which are well known to the person skilled in the art.

Also the term “diagnostic composition” relates to a composition, optionally for administration to a patient, preferably a human patient and may comprise the essentially same additional compounds as discussed in connection with the pharmaceutical composition. While pharmaceutical compositions are to cure or prevent a disease a diagnostic composition is to identify the presence and optionally also the site of a disease in a subject. The diagnostic composition preferably comprises a diagnostically active compound as discussed herein above, such as a radiolabel or a fluorophore.

The various components of the kit may be packaged into one or more containers such as one or more vials. The vials may, in addition to the components, comprise preservatives or buffers for storage. The kit may comprise instructions how to use the kit, which preferably inform how to use the components of the kit for diagnosing a tumor and/or for grading a tumor and/or for tumor prognosis.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The Figures show.

FIG. 1. Modeling and design strategy of Conformationally-adaptive Peptide BioSensor (CaPSens). (A) Schematic of targeting binding and activation residues for design of chemotactic receptors with enhanced responses towards peptide attractants. The peptide ligand makes specific contacts with receptor pocket residues that are classified as drivers of binding or activation. Through design, receptor:peptide connectivity can be rewired to promote binding (top), activation, or both (bottom) to ultimately reprogram the cell migratory response. (B) Pipeline of the design strategy involving receptor:peptide modeling, rational design, experimental validation, and selection of final model ensemble.

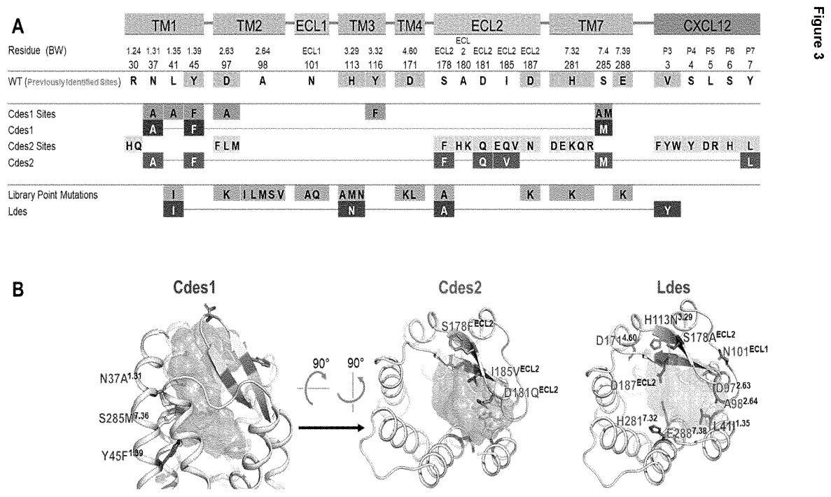

FIG. 2. General overview of the protein-peptide binding design strategies employed in the study. (A) Schematic view of a conformational energy landscape describing the binding of a flexible peptide to a receptor. The peptide is represented with 6 light grey spheres and adopts distinct conformations in each local energy minimum. (B) A conventional design approach by conformational selection stabilizes one favored receptor:peptide conformation, while destabilizing others. Destabilizing interactions are represented as steric clashes. (C) The novel design approach as described herein to preserve dynamism selects amino-acid substitutions stabilizing multiple receptor:peptide conformations, hence maintaining conformational entropy at the binding interface.

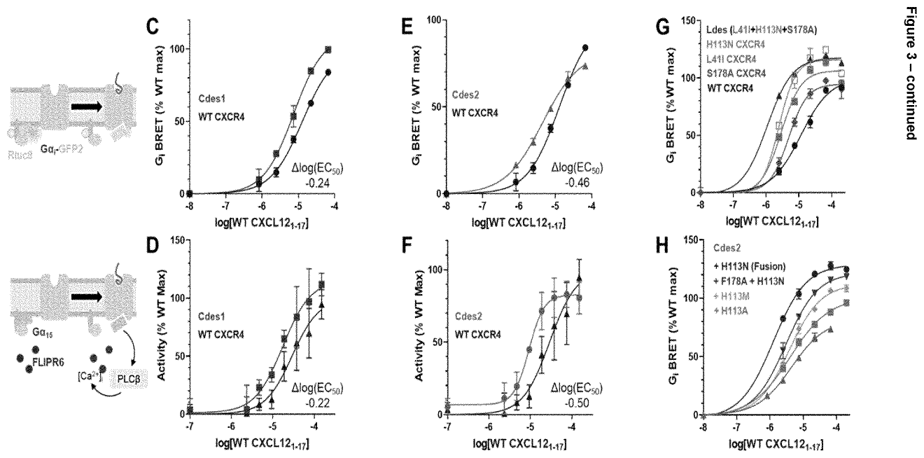

FIG. 3. Design of receptor peptide binding sites for enhanced sensing. (A) Primary sequence mapping of receptor residues targeted for design. (B) Location of the designed residues (shown in sticks) mapped onto the biosensor peptide binding site backbone structure (shown in cartoon). The peptide is represented as a gray-colored surface. (C-H) Peptide-induced cell signaling responses of designed receptors measured through Gi activation and calcium release: Gi BRET of Cdes1 design (C), Cdes2. design (D), and library-screened mutations (E). Calcium mobilization of Cdes1 design (F) and Cdes2 design (G). Effect of single-point library mutations in Cdes2 design background (H).

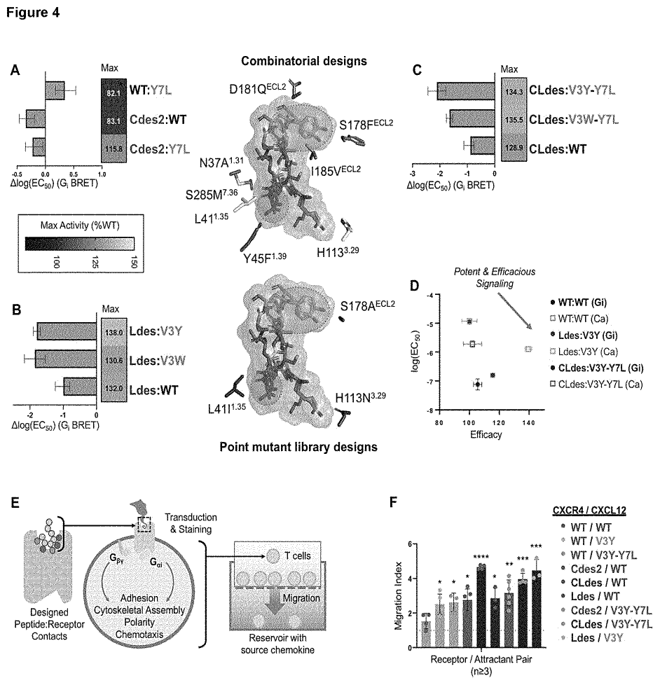

FIG. 4. Design of highly-sensitive and chemotactic receptor-peptide pairs. A-D. Shifts in sensitivity (95% confidence interval of dose response curve fits) and maximum activity (fitted value) for various receptor-peptide pairs involving the following designed peptides: (A) CXCL12 Y7L variant, (B) CXCL12 V3 substitutions, (C) CXCL12 V3Y/W-Y7L. (D) Changes in potency and efficacy across three separate experiments (each n=3), s.e.m. plotted. (E) Schematic of boyden chamber migration assay of T cells transduced with engineered receptors and (F) migratory responses of transduced primary T cells towards full-length chemokine. Bars are colored according to the transduced CXCR4 variant, and individual points are colored according to the CXCL12 variant. *: p≤0.05, **: p≤0.01, ***: p≤0.001, ****: p≤0.0001.

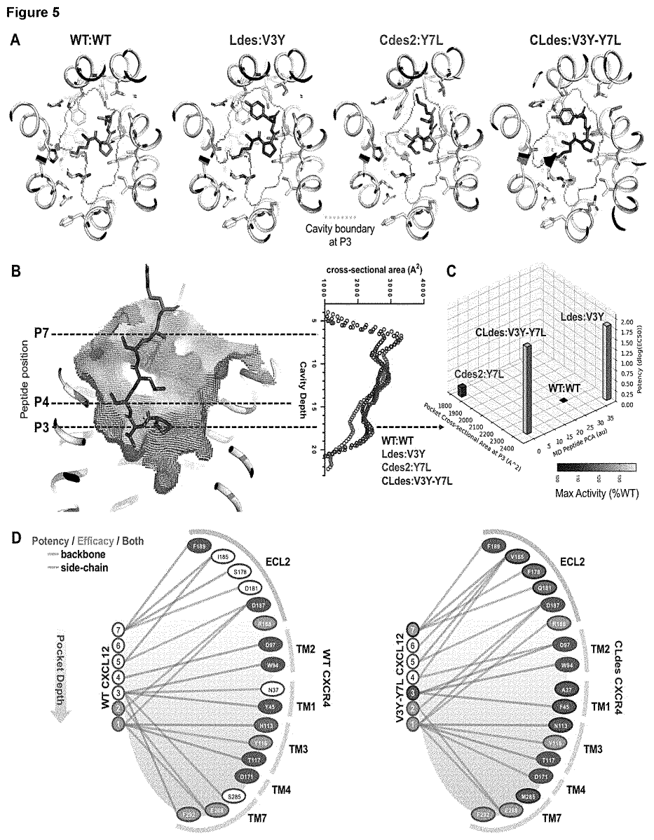

FIG. 5. High level of structural adaptation at the designed peptide-receptor binding interface. (A) Cross-sectional views of receptor cavity at CXCL12.P3 depth for the following pairs: WT CXCR4:WT CXCL12, Ldes CXCR4:V3Y, Cdes2 CXCR4:V3Y-Y7L CXCL12, and CLdes CXCR4:V3Y-Y7L CXCL12. The solvent accessible boundary at the P3 peptide position is shown in yellow. (B) Receptor cavity cross-sections at 3 distinct peptide positions mapped onto the WT CXCR4: WT CXCL12 structure (left). Cross-sectional area of the binding cavity as a function of cavity depth for distinct peptide-receptor pairs (right). (C) 3D structure-activity map. Activity shifts from WT of individual receptor-peptide pairs (z axis for potency and bars colored according to maximal activity) are plotted as a function of conformational shifts of the peptide (x axis: calculated by Principal Component Analysis on bound peptide ensembles (see Methods)) and conformational shifts of the receptor binding pocket (y axis: calculated by cross sectional area (see Methods)). (D) Peptide:receptor contributions to potency and efficacy were derived from previous mutagenesis studies (Wescott, 2016; Kufareva, 2014; Thiele, 2014; Crump, 1997) and this study for the WT CXCR4:CXCL12 and designed complexes, respectively. Residue connectivity inferred from direct contacts in the final WT CXCR4:WT CXCL12 and CLdes CXCR4:V3Y-Y7L CXCL12 structures.

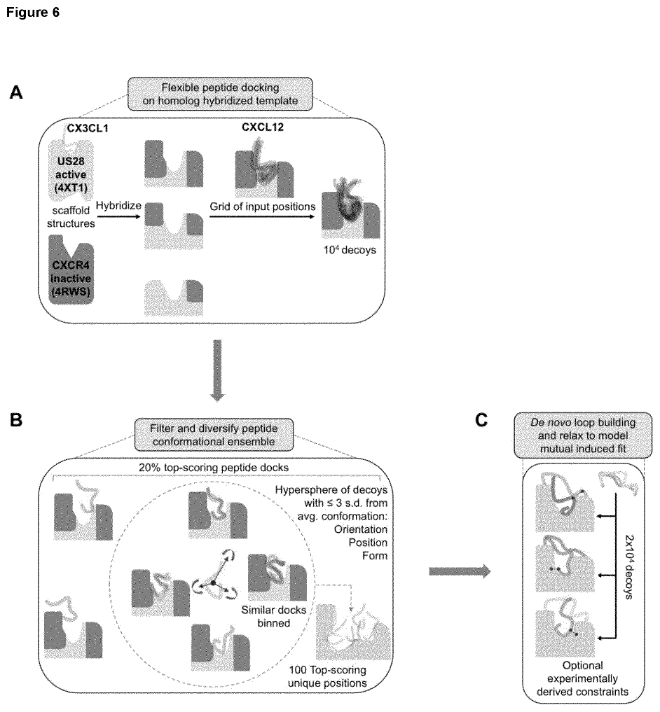

FIG. 6. Generation of an ensemble of diverse peptide-binding scaffold structures. (A). Scaffolds were hybridized between active-state US28 bound to CX3CL1 (4XT1) and inactive-state CXCR4 bound to a chemokine antagonist (4RWS). N-terminal peptide residues of CXCL12 were threaded onto CX3CL1 and flexible peptide docking was started from a grid of starting positions covering the cavity volume. (B). Peptide decoys are filtered by binding interface score and 100 diverse peptide poses are selected for uniqueness across three features: orientation, position, and form. (C). Subsequent loop modeling is performed with optional experimentally derived constraints and the resulting bound complexes are extensively relaxed to model mutual induced fit effects.

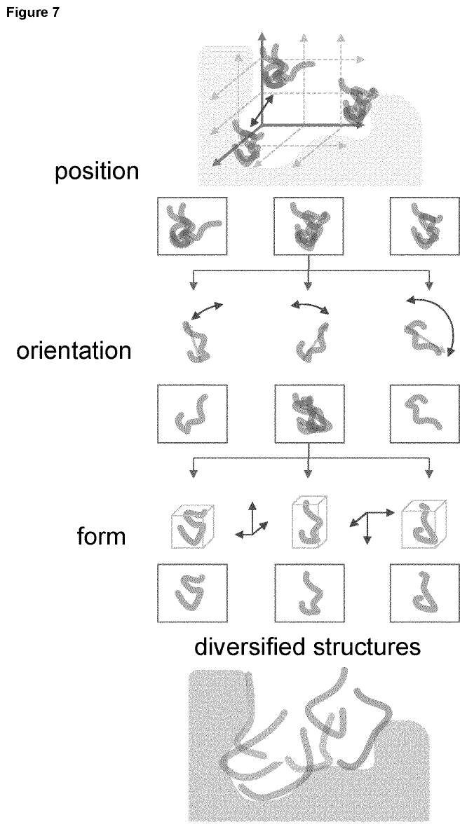

FIG. 7. Peptide conformation selection through diversification. Schematic description of how peptide conformations are selected through the novel diversification algorithm as described herein. The decoys are aligned along the receptor structures and the Ca coordinates of the peptide poses were stored in a matrix, whose principal axes are calculated in reference to the common principal axes of the receptor. Each peptide pose is binned in 3 sequential steps for the following features: (1) peptide position relative to the receptor by center of mass of the peptide. (2) peptide orientation by the angles of the peptide's 3 principal axes in reference to the 3 principal axes of the receptor. (3) internal peptide form by the eigenvalues of the matrix of the Ca-coordinates of the peptide. For each bin, the decoy with the best combined interface and peptide energy score is selected for loop rebuilding and relaxation.

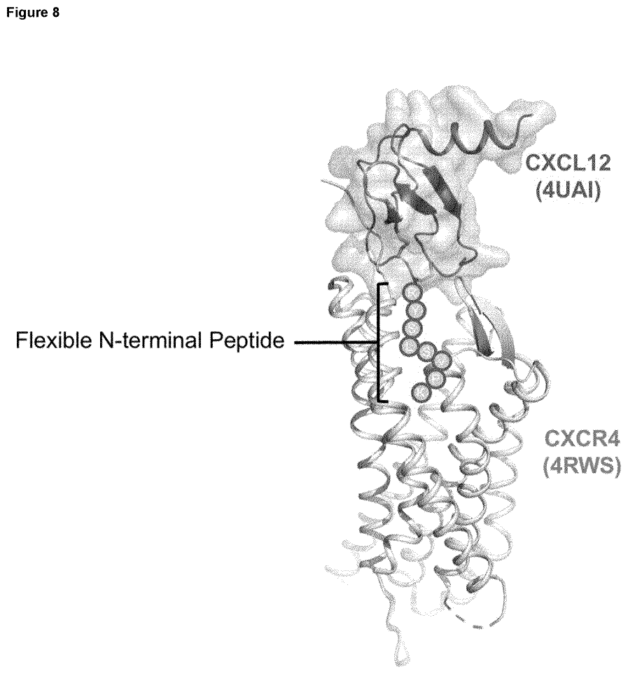

FIG. 8. General CXCR4:CXCL12 architecture. The core chemokine domain of CXCL12 (4UAI) is aligned to the chemokine antagonist complexed with CXCR4 from the 4RWS crystal structure. Flexible N-terminal residues, which are the focus of modeling and design in this study, are depicted in circles.

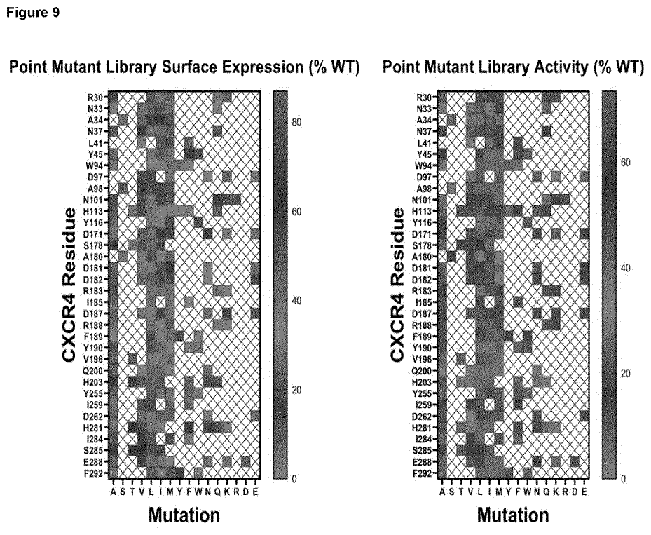

FIG. 9. Computationally-guided point mutant library activity screen. Left: Receptor surface expression. Right: Calcium mobilization activity at 20 nM CXCL121-17 peptide.

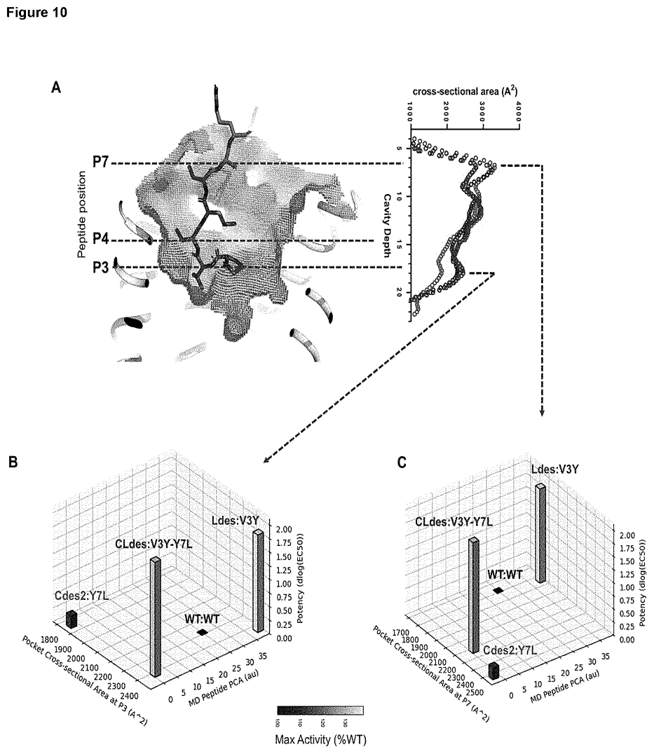

FIG. 10. 3D structure-activity maps of the receptor-peptide pairs. (A). Receptor cavity cross-sections at 3 distinct peptide positions mapped onto the WT CXCR4: WT CXCL12 structure (left). Cross-sectional area of the binding cavity as a function of cavity depth for distinct peptide-receptor pairs (right). (B) 3D structure-activity map. Activity shifts from WT of individual receptor-peptide pairs (z axis for potency and bars colored according to maximal activity) are plotted as a function of conformational shifts of the peptide (x axis: calculated by Principal Component Analysis on bound peptide ensembles (see Methods)) and conformational shifts of the receptor binding pocket at P3 (y axis: calculated by cross sectional area (see Methods)). (C) Same as (B) with conformational shifts of the receptor binding pocket at P7.

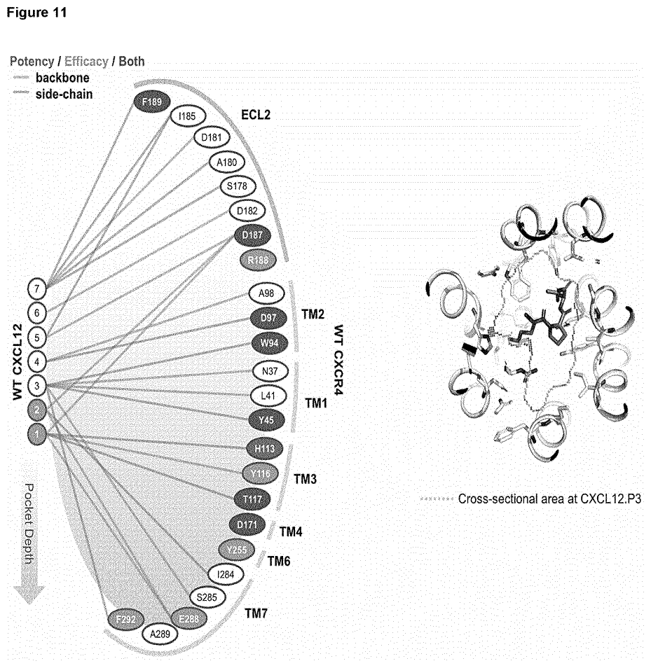

FIG. 11. Full residue connectivity inferred from direct contacts in final WT CXCR4:WT CXCL12 model. Peptide:receptor contributions to potency and efficacy derived from previous mutagenesis studies (Wescott, 2016; Kufareva, 2014; Thiele, 2014; Crump, 1997) and designs from this study. Cross-sectional area of binding cavity shown at right.

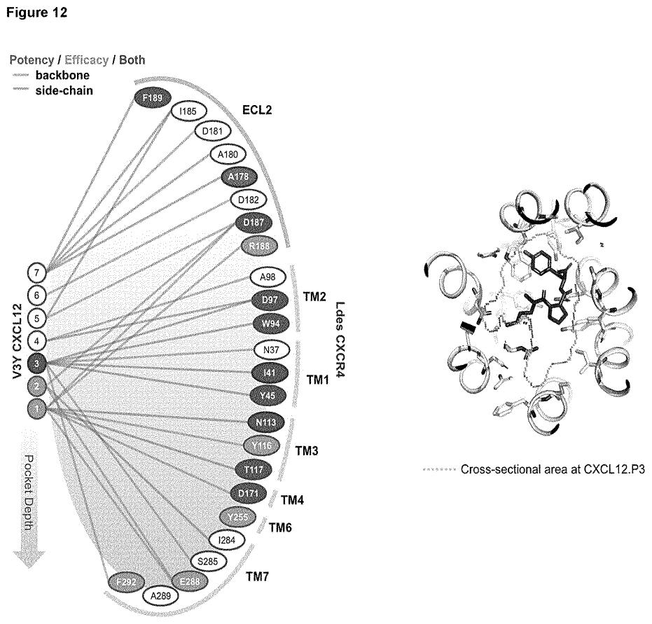

FIG. 12. Full residue connectivity inferred from direct contacts in final Ldes CXCR4:V3Y CXCL12 model. Mutated residues outlined in purple. Peptide:receptor contributions to potency and efficacy derived from previous mutagenesis studies (Wescott, 2016; Kufareva, 2014; Thiele, 2014; Crump, 1997) and designs from this study. Cross-sectional area of binding cavity shown at right.

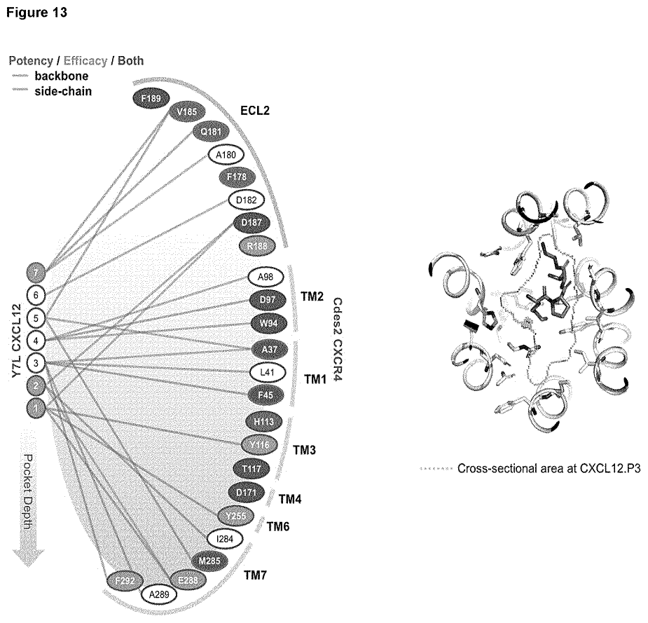

FIG. 13. Full residue connectivity inferred from direct contacts in final Cdes2 CXCR4:Y7L CXCL12 model. Mutated residues outlined in green. Peptide:receptor contributions to potency and efficacy derived from previous mutagenesis studies (Wescott, 2016; Kufareva, 2014; Thiele, 2014; Crump, 1997) and designs from this study. Cross-sectional area of binding cavity shown at right.

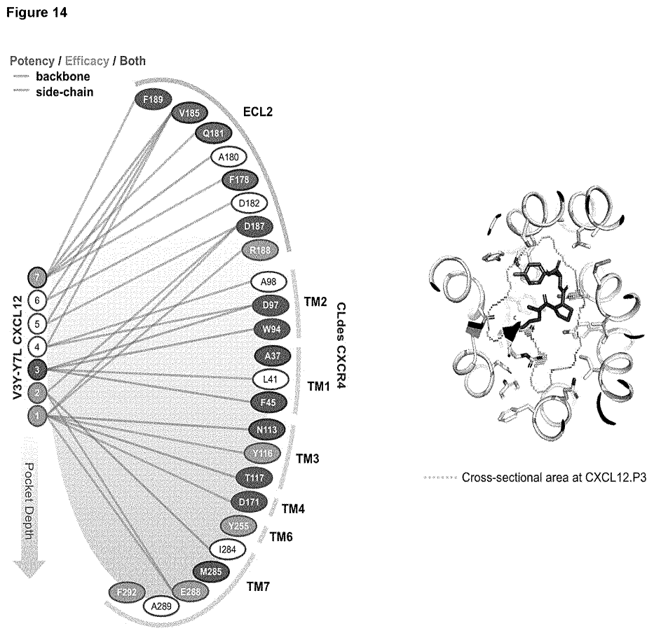

FIG. 14. Full residue connectivity inferred from direct contacts in final CLdes CXCR4:V3Y-Y7L CXCL12 model. Mutated residues outlined in dark red. Peptide:receptor contributions to potency and efficacy derived from previous mutagenesis studies (Wescott, 2016; Kufareva, 2014; Thiele, 2014; Crump, 1997) and designs from this study. Cross-sectional area of binding cavity shown at right.

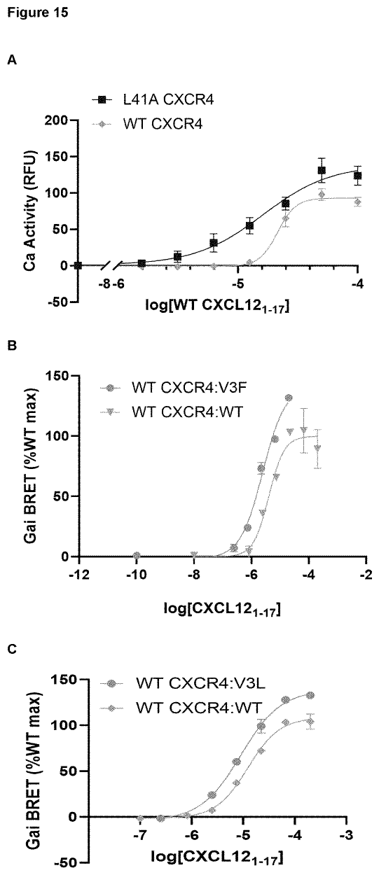

FIG. 15. Peptide-induced cell signaling responses of point mutations. (A) Enhanced calcium release of the L41A CXCR4 variant. (B) Enhanced Gi activation by the V3F CXCL12 variant. (C) Enhanced Gi activation by the V3L CXCL12 variant.

The examples illustrate the invention.

### EXAMPLE 1: RESULTS

Here, a computational strategy is described and applied for engineering membrane receptors with high binding sensitivity to flexible peptide ligands and potent allosteric signal transduction responses across the membrane. Unlike previous work that only optimize binding and model receptors as rigid target structures⁹, fully flexible receptor:peptide conformational ensembles were build that enable the precise modeling of signaling active states and the design of complexes with novel contact networks enhancing both binding sensitivity and allosteric response (FIG. 1A). Through this approach, it is aimed to design custom-built modular biosensors that can link binding of a flexible peptide input signal to selective, fine-tuned and complex cellular responses through genetically encoded single-receptor domains. This new class of biosensors is defined as CaPSens, which stands for Conformationally-adaptive Peptide BioSensor.

To demonstrate this strategy, ultrasensitive CaPSens of chemotactic peptides were designed for reprogramming cellular migration (FIG. 1A). Chemotactic peptides are attractive targets since directional movement of cells in response to gradient of these molecules (e.g. chemotaxis) is essential throughout biology and represents one of the great challenges of synthetic cell biology. For example, efficient immune cell homing to cancer cells is one of the bottlenecks in modern immunotherapy¹⁰⁻¹⁴. Hence, these therapeutic approaches would benefit from engineered cytotoxic lymphocytes with enhanced chemotaxis towards tumor sites. Since cell migration relies on the precise orchestration of diverse intracellular activities, novel synthetic sensing properties w3re build and carved onto natural chemotactic receptor scaffolds for optimal interfacing with the complex intracellular machinery of eukaryotic cells that mediate chemotaxis¹⁵⁻¹⁸.

Molecular recognition between flexible peptide and signaling receptors usually involves significant structural rearrangements of both molecules through conformational selection (i.e. selection from an ensemble of unbound conformations) and induced fit (i.e. conformational changes occurring upon binding) effects. Therefore, it was reasoned that an effective method for evolving novel interaction networks optimizing peptide recognition and long-range allosteric response should explore a vast conformational binding space through sampling of peptide conformational ensemble but also extensive structural relaxation of peptide bound receptor complexes. The computational strategy was developed with these ideas in mind and proceeds in the following main steps (Methods, FIG. 1B, FIG. 2, FIG. 6): (i) building hybrid transmembrane (TM) scaffolds in active signaling conformation using structure parts from distinct chemotactic receptors to generate diverse possible biosensor templates; (ii) de novo docking of fully flexible peptides onto the receptor scaffold binding sites to identify possible interacting conformations from the large diversity of unbound peptide structures; (iii) filter and diversify peptide positions to generate a peptide-bound receptor ensemble representative of the vast conformational binding space and diverse networks of contacts; (iv) de novo loop rebuilding of the biosensor scaffold to accommodate peptide-bound structure; (v) relax the resulting receptor:peptide complex structure to populate the most optimal binding conformations through mutual induced fit effects; (vi) computational selection of novel binding and allosteric contact networks at the receptor:peptide interface followed by structural relaxation; (vii) experimental validation of selected designs using a battery of cell-based functional assays; (viii) experimentally guided refinement of receptor:peptide conformational ensemble to improve modeling accuracy; (ix) design of highly sensitive receptor:peptide super-agonist pairs for enhanced chemotaxis.

As a proof of concept, peptide ligand agonists were modeled and designed starting from the N-terminal partially unstructured agonist region of the chemokine CXCL12 (FIG. 8), which promotes strong activation of the CXCR4 receptor¹⁹⁻²⁰. To build and evolve CaPSens scaffolds sensing CXCL12-derived peptides, structural parts from the chemokine receptor family were selected. In absence of a CXCR4 structure in the signaling active form, biosensor templates were assembled from local structures of CXCR4 in the inactive form and the structure of the homologous viral chemokine receptor US28 bound to CX3CL1 (4XT1)²¹, the only active-state chemokine receptor structure available at the time of modeling. The modeling stage (steps i-iv) yielded 9 major peptide-bound biosensor scaffold structures that provided starting templates for a first round of computational design (Methods, FIG. 1B). In the following, designs are named by the approach (Cdes for combinatorial design, Ldes for point mutant library design, CLdes for combined Cdes and Ldes solutions) and design generation (1 and 2).

Since the first two N-terminal positions of CXCL12 are critical for activation and even conservative mutations can lead to drastic signaling defects²²⁻²⁴, the initial computational design was focused on improving the binding of the sensor to positions P3 through P8 of the CXCL12-derived peptide, up to the CXC motif. The first round of calculations yielded a novel binding hotspot motif with improved interfacial contact density between the TM1/7 interface and P3 of the peptide as well as new interactions with the allosteric position 1.39 (FIG. 3A,B). The peptide binding and signaling properties of the Cdes1 receptor were validated in HEK cells using cell based assays reporting G-protein Gi activation and Ca^{2 +} mobilization that are triggered by native chemokine receptors and known to be crucial for chemotactic responses. Consistent with the prediction, the designed receptor displayed enhanced sensitivity to CXCL12 (FIG. 3C,D). It was built upon the initial success of the Cdes1 design by further optimizing the binding interface upstream of P3. A second binding hotspot motif was selected between P7 of CXCL12 and 3 positions lining the beta hairpin of the receptor second extracellular loop (ECL2) (FIG. 3A,B). Combining the 2 designed motifs into the Cdes2 receptor led to substantially enhanced potency in calcium mobilization (3.1-fold over WT) and G-coupling (3.2-fold over WT) (FIG. 3E,F). To rapidly identify additional binding and activating motifs, a computationally guided library of variants built from the initial ensemble of receptor:peptide models was then rationally created and screened. Each variant was designed by mutating a single predicted peptide binding and/or allosteric residue and assayed for calcium mobilization (FIG. 9) and Gᵢ coupling (FIG. 3G). Activating point mutations were identified at novel sites on TM1, TM3 and ECL2 and assembled into a library-selected combination (Ldes) receptor variant (FIG. 3A,B; FIG. 8; FIG. 15A). The Ldes design was considerably more sensitive than the starting CXCR4 WT scaffold with close to 11-fold enhanced Gi potency and 120% higher efficacy (FIG. 3G). it was next thought to combine the initial designed binding hotspot motifs from Cdes2 with these new activating sites. While the positions on TM1 and 7 strongly overlapped, the substitutions at position 113^{3,29} occupy a region of the binding pocket not exploited by the Cdes1 or Cdes2 designs. Additive effects were observed when combining the most activating H113N^{3,29} mutation with the Cdes2 design and led to a CLdes sensor that had the second most potent and sensitive Gi response (more than 9-fold increase over WT) against WT CXCL12 in the designs (FIG. 3H). These results indicate that the approach provided herein can readily design highly sensitive sensors of the WT CXCL12 chemokine derived peptide by optimizing both binding and signaling determinants.

It was next sought to create selective receptor:peptide pairs by designing novel peptide super-agonists. Such synthetic sensor-response systems would provide orthogonal solutions for reprogramming cellular activity and bypass the high level of binding promiscuity inherent to native receptors. From the computational models, 2 sites P3 and P7 were identified on the peptide scaffolds where novel and stronger contacts with the receptor binding pocket could be designed. A designed Leu at P7 further optimized packing complementarity with the binding hotspot motif of the Cdes2 design, enhanced Gi efficacy by 130% of the designed sensor while decreasing the overall response of the WT receptor scaffold (FIG. 4A). At position P3, the calculations identified bulky aromatic residues predicted to complement I41^{1,35} and W94^{2,60} at the TM 1/2 pocket interface of the designed sensors, leading to extremely powerful activating effects. Specifically, the Ldes:V3Y peptide pair displayed more than 80-fold enhanced potency and a 125% increase in efficacy compared to the WT receptor:peptide pair (FIG. 4B,D; FIG. 15B,C). The CLdes:V3Y-Y7L peptide pair boosted both potency and efficacy by more than 100-fold and 134%, respectively (FIG. 4C,D). These results demonstrate the power of the computational approach as provided herein for engineering novel synthetic receptor:peptide pairs with highly sensitive binding properties and potent downstream signaling.

It was next assessed whether the ultra-sensitive CaPSens also elicited a cell migratory phenotype with concomitant sensitivity upon detection of chemotactic chemokines. Chemotaxis results from the complex orchestration of multiple intracellular pathways that control receptor oligomerization, cell motility, polarity, adhesion, following receptor-mediated G-protein activation triggered by the sensing of chemokine proteins¹⁵⁻¹⁸ (FIG. 4E). Such validation represents a stringent test of the ability to leverage molecular design for cell engineering and reprogram complex cellular behaviors in responses to environmental cues. Primary human T cells were transduced with selected designed sensors and measured their migration against gradients of full-length WT or engineered chemokines incorporating the designed N-terminal peptide tail. Chemotaxis was measured using boyden chambers in which cells can migrate across a porous membrane towards a reservoir containing chemoattractant. Migratory indices were measured as a function of the fold-migration over a no-chemoattractant control for each transduced donor. T cells transduced with the Cdes2, Ldes and CLdes designs displayed up to almost 5-fold increased migration towards 100 nM wild-type CXCL12. At this level of chemoattractant, WT CXCR4 promoted around 1.5-fold enhanced migration when compared to the no-attractant controls (FIG. 4F). The engineered CaPSens also boosted T cell migration by up to 4.5-fold when exposed to designed chemokines (FIG. 4F). The enhanced cellular migration demonstrates that the designed molecular signaling properties leading to ultra-high sensitivity of the sensors translate into the intended reprogramming of cell phenotypes. It also indicates that the strategy focusing on the flexible peptide region of the chemokine is generalizable to the design of biosensors responding to full-length chemoattractants. Overall, the results suggest that engineered receptors could trigger migration towards cancer-prone sites at longer distances with shallower chemokine gradients when compared to native chemotactic systems. The designed CaPSens:hyper-agonist peptide pairs open the door to bringing cell migration under exogenous and spatiotemporal control, providing a promising new synthetic cell biology tool.

The diverse designed receptor-peptide agonist pairs offer a unique opportunity to assess the structural underpinnings of receptor:peptide binding and agonism. Unlike most binding interfaces between globular proteins, the designs displayed considerable structural adaptation to sequence changes. On the peptide side, large shifts were observed in peptide backbone and side-chain conformations except for the two most buried and constrained P1 and P2 sites (FIG. 5A). Structural remodeling of the receptor were also noticeable and best quantified using a volumetric analysis of the peptide binding site. Cross-sectional areas at different depths of the binding pocket highlighted significant conformational adaptation of the binding surface (by up to 25% at certain cavity depths) in response to the different peptide conformation and sequences (FIG. 5B). When mapped on a 3D structure-function relationship map (FIG. 5C, FIG. 10), the three most sensitive designed pairs occupy subspaces that are far apart in both receptor binding site and peptide conformation dimensions. These observations suggest the existence of multiple solutions for designing potent peptide biosensors with optimal sensing and signaling response. Owing to substantial backbone movements, the peptide may access an ensemble of microstates whose occupancy can be readily shifted via changes in sequence and/or receptor shape. The receptor binding pocket displays also significant structural plasticity and can accommodate diverse peptide conformations. This concurrent structural adaption on both sides of the interface underlies a design process by mutual induced fit that enables the formation of diverse novel networks of productive binding and allosteric contacts, respectively modulating the potency and efficacy of the signaling response (FIG. 5D, FIGS. 11-14). In fact, each of the highly sensitive biosensor-peptide pair displayed specific patterns of potency and efficacy determining contacts (FIGS. 11-14). For example, designed receptor allosteric residues such as N113 interacting with the key agonistic P1 site tend to enhance both efficacy and potency while distal binding residues contacting P7 mostly affected potency. More indirectly, through rewired P7 interactions with the ECL2 loop in the CLdes-V3Y-Y7L peptide design pair, the P7 position was unlocked from its native conformation enabling the peptide to populate a more efficacious microstate through significant backbone shifts. The results as provided herein support a view of receptor-peptide sensing where the inherent plasticity of the binding interface enables the efficient adaptation of contact networks in response to even limited shifts in sequence space. Modeling and designing receptor-peptide interactions as conformationally dynamic complexes allows us to exploit this hallmark and readily evolve novel functional pairs.

To the best knowledge of the inventors, the computational design of peptide binding receptors with highly optimized binding and allosteric signaling functions is unprecedented. Most biosensor design approaches have focused on engineering protein domains for optimal recognition of structurally well-defined molecules. By targeting flexible and structurally uncharacterized peptides, design platform as provided herein significantly expands the range of molecules that can be detected by biosensors. Unlike approaches that rely on multi-domain sensor reconstitution upon ligand sensing, method as provided herein optimizes the coupling between molecular recognition and allosteric response in a single protein domain and can generate CaPSens with unprecedented dynamic and sensitive responses. Carving biosensors into versatile GPCR scaffolds offers key additional advantages. GPCRs can now be engineered to trigger a wide range of intracellular functions through reprogrammed coupling to diverse effectors including G-proteins and arrestins^{25,26}. Alternatively, inserting fluorescent protein domains into GPCR scaffolds enables fast and direct optical detection of ligand molecules²⁷. As such, the approach as provided herein paves the road for a wide range of synthetic biology, diagnostics and therapeutic applications that would benefit from sensor systems that trigger complex cellular outputs or enable direct highly sensitive detection of chemical cues.

### EXAMPLE 2—METHODS

### CXCR4-CXCL12 Active-State Modeling

### Constructing Initial Chemokine Receptor Scaffolds

The initial goal was to build CXCR4-based receptor scaffolds in the active signaling state for engineering precise interactions with peptide agonists that promote strong binding and potent response. In absence of a CXCR4 active state structure, hybrid scaffolds were generated using elements from the inactive-state structure of CXCR4 crystallized with a viral chemokine antagonist (4RWS) structure²⁸, and the active-state structure of the viral chemokine US28 receptor crystallized with CX3CL1 (4XT1)²¹. The hybridization aimed to incorporate the maximal number of active state structural features from 4XT1 while preventing significant de novo reconstruction of the transmembrane core region due to poor sequence-structure alignment between the viral chemokine template and the CXCR4 sequence. Hybridized scaffolds incorporated structural elements of either 4RWS or 4XT1 around the peptide binding pocket in ECL2 (residues 87-101) and the extracellular head of transmembrane helix (TM) 2 (residues 174-192), local regions that differ significantly between both templates (FIG. 1B, FIG. 6). Subsequently, the CXCR4 sequence was threaded onto the hybrid template structures guided by sequence-structural alignments using HHpred (US28 and CXCR4 share 29% sequence identity overall)^{29,30}. Prior to hybridization, the 4RWS template structure was corrected back to wild-type sequence (C187D/W125L) and missing loops in ICL1 and the truncated ICL3 were repaired by loop remodeling (cyclic coordinate descent algorithm)^{31,32}. 1000 decoys were generated and clustered and the lowest energy cluster was selected for further modeling.

### Peptide Docking

The N-terminal tail of CXCL12 in experimental structures of the chemokine is often too disordered or lack receptor context to truly represent an active-state conformation. Thus the sequence of that region was threaded onto the active-state structure of CX3CL1 in complex with US28 (4XT1) to generate a starting template for subsequent flexible docking. The N-terminal 11 residues of CXCL12, including the CXC motif, were aligned to the docked position of CX3CL1 in the active-state structure. The N-terminal CXCL12.K1 was aligned to the H2 position of CX3CL1 to match the partial positive charge of the imidazole ring since the Q1 residue of CX3CL1 is cyclized to form pyroglutamate to produce a neutral N-terminus²¹. (Alternately, CXCL12.K1 was aligned to H3 of CX3CL1, but docking from this initial position yielded models with weak interface energies and few contacts to key binding residues). That initial peptide position was translated across the receptor pocket in a cubic grid around the aligned position and prepacked to generate 9 starting positions for subsequent flexible peptide docking⁹. 10,000 decoys were generated from the 9 unique starting inputs. In addition to unconstrained docking, different sets of constraints were used to enrich the following putative receptor:peptide interactions that represent known critical agonistic contacts: CXCR4.D97-CXCL12.K1 ε-amine, CXCR4.D97-CXCL12.S4, CXCR4.D171-CXCL12.K1 ε-amine, CXCR4.E288-CXCL12.K1 ε-amine, CXCR4.E288-CXCL12.N-term amine, and a tripartite constraint set for CXCR4.D97-CXCL12.S4+CXCR4.D171-CXCL12.K1 ε-amine+CXCR4.E288-CXCL12.N-term amine.

### Loop Rebuilding and Ensemble Relaxation to Model Mutual Induced Fit Effects

The C-alpha coordinates of each of the 20% lowest energy peptide poses were stored in a matrix and described by three features: (1) peptide position by center of mass of the peptide (x, y, z), (2) peptide orientation by rotational angles between principal axes of eigenvectors (θₓ, θ_{y}, θ^{z}), (3) internal peptide conformation by eigenvalues (e₁, e₂, e₃). Peptide poses were filtered to remove decoys that are more than 3 standard deviations of the average value for each feature. A hypersphere radius (Δx²+Δy²+Δz²+Δθₓ², Δθ_{y}², Δθ_{z}²+Δe1²+Δe2²+Δe3²) was then defined such that at least 100 diverse peptide positions could be identified that differ by more than the hypersphere radius for each of the 3 features (FIG. 7).

For each diversified docked position, de novo loop modeling was performed to build loop structures onto the initial scaffolds that best accommodate the bound peptide conformation (200 decoys per diversified input). To further capture and model peptide induced fit effects on the receptor structure, receptor:peptide complexes were subsequently relaxed over all conformational degrees of freedom. Receptor structures were restrained using distance constraints derived from sequence conservation²⁶. The 10% lowest interface energy decoys were clustered by structural similarity of the peptide conformation and key binding residues in the receptor pocket. Convergent clusters (RMSD of top 5 cluster members ranged from 0.2 to 1.0 Å) were selected by interface energy and satisfaction of the experimentally informed constraints used in peptide docking. 9 representative models were selected for the design of novel receptor-peptide complexes (FIG. 7).

### Refinement of the Ensemble of CXCR4-CXCL12 Active-State Models

While the initial set of WT CXCR4-CXCL12 models provided key input scaffold structures for engineering novel functional peptide-receptor interactions, not every model was expected to accurately represent the receptor-peptide conformational ensemble. The initial set of models was filtered and refined to find an ensemble of flexible peptide dock positions that best recapitulate the observed mutational effects and increase overall prediction accuracy. The cluster of models in best support of observed changes in EC₅₀ was used as an initial input for further flexible peptide docking refinement to identify optimal conformations for the WT, Cdes2, library-selected and CLdes designs. A single constraint was enforced for electrostatic interaction between E288^{7,39} of CXCR4 and the ε-amine of CXCL12.K1. Top-scoring cluster members then underwent another round of side-chain repacking and energy minimization without constraints. The interface energies of the resulting models were again validated against observed changes in EC₅₀ to identify conformational states representative of an ensemble of peptide positions which largely support the designed effects measured experimentally.

### Molecular Dynamics Simulations

To further confirm the above selected models and analyze the structural diversity of the designed binders using an orthogonal approach, the receptor-peptide conformational binding space was sampled using MD simulations in explicit lipid bilayers. The final selected models for WT:WT, Ldes:V3Y, Cdes2:Y7L, and CLdes:V3Y-Y7L CXCR4:CXCL12 complexes were used as starting input poses for MD simulations. The receptor-ligand complex was inserted into a regular hexagonal POPC lipid bilayer with 90 Δ perpendicular distance between any parallel sides and solvated by 22.5 Δ layer of water above and below the bilayer with 0.15 M of Na⁺ and Cl⁻ ions using CHARMM-GUI bilayer builder^{33,34}. Simulations were performed with GROMACS 2020.5^{35,36} with CHARMM36 forcefield³⁷ in an NPT ensemble at 310K and 1 bar using a Nosé-Hoover thermostat (independently coupled to three groups: protein, membrane, and solvent with a relaxation time of 1 ps for all three) and Parrinello-Rahman barostat (with semi-isotropic coupling at a relaxation time of 5 ps) respectively. Equations of motion were integrated with a timestep of 2 fs using a leap-frog algorithm. Each system was energy minimized using a steepest descent algorithm for 5000 steps, and then equilibrated with the atoms of the ligand-receptor complex and lipids restrained using a harmonic restraining force in 6 steps as shown in the table below:

| Step | 1 | 2 | 3 | 4 | 5 | 6 |
|---|---|---|---|---|---|---|
| Time [ps] | 125 | 125 | 125 | 250 | 250 | 250 |
| Timestep [fs] | 1 | 1 | 1 | 2 | 2 | 2 |
| Ensemble | NVT | NVT | NPT | NPT | NPT | NPT |
| Pos restraints | | | | | | |
| [kJ/(mol · nm)] | | | | | | |
| Backbone | 4000 | 2000 | 1000 | 500 | 200 | 50 |
| Side chain | 2000 | 1000 | 500 | 200 | 50 | 0 |
| Lipid & ligand | 1000 | 400 | 400 | 200 | 40 | 0 |
| Dih restrains | 1000 | 400 | 200 | 200 | 100 | 0 |
| [kJ/(mol · nm)] | | | | | | |

After constrained equilibration, five replicas of 200 to 300 ns (as shown in the table below) were run for each system. The first 50 ns of the simulations were discarded as time needed for the system to equilibrate, as shown by the Ca RMSD of the receptors and the ligands.

| | | Simulated time | Total simulated |
|---|---|---|---|
| System | Number of runs | of every run | time |
| WT CXCR4: WT | 5 | 3 × 200 ns | 1.2 μs |
| CXCL12 | | 2 × 300 ns | |
| Ldes: V3Y | 5 | 2 × 200 ns | 1.3 μs |
| | | 3 × 300 ns | |
| CLdes: V3Y-Y7L | 5 | 4 × 200 ns | 1.1 μs |
| | | 1 × 300 ns | |
| Cdes2: Y7L | 5 | 5 × 300 ns | 1.5 μs |

### Principle Component Analysis (PCA) of Bound Peptide Conformational Ensemble

PCA was performed on the cartesian coordinates of Cα and Cβ atoms of peptide ligands from receptor: peptide conformations selected by combining replica molecular dynamics trajectories from all the studied systems. Representative models from the molecular dynamics trajectories were chosen as the highest density points in the space of principal components (PCs) 1 and 2. The first 2 PCs explain 39.9% and 20.8% of the variability of the data respectively.

### Engineering CXCR4-CXCL12 Complexes for Enhanced Activity

### Computational Combinatorial Design

Designable sites were identified on both the peptide and receptor sides of the different binding interfaces featured in the initial set of 9 CXCL12-bound receptor WVT models. Novel combination of amino-acids and conformations were searched concurrently for improving receptor-peptide association and signaling response. In silico mutagenesis was performed as previously described³⁸, allowing all possible residue substitutions at designable sites and selecting top scoring models for interface energy improvement from WVT among 200 independent trajectories, such that scores converged for the top 10% models. All residues with heteroatoms within 5.0 Δ of any designable residue were repacked and their backbone and side-chain minimized. Cdes2 designs were made on 2 different clusters of models that showed good agreement with the initial Cdes1 design. Designs were computationally validated by peptide docking refinement (10,000 independent trajectories) to identify the optimal docked peptide position at the binding interface of the designed complexes and refine the binding energy predictions. The 10% lowest energy decoys were verified by RMSD to the intended design position, cluster size, and interface energy after repacking.

### Computationally-Guided Point Mutant Library

A computationally guided library of variants was built from the initial ensemble of receptor:peptide models. Each variant was designed by mutating a single predicted peptide binding and/or allosteric residue. The mutant library consisted of substitutions involving modest changes in sidechain size, and polarity that would largely be compatible with the ensemble of initial receptor:peptide conformations.

The following mutations were included in the library: R30A/K/Q/L/I/M, N33A/Q/V/L/I/M, A34S/V/L/I/M, N37A/Q/V/L/I/M, L41I/V/F/M, Y45A/F/L/I/M/W, W94A/Y/F/L/I/M, D97E/N/V/L/M/K, A98S/V/L/I/M, N101A/Q/V/L/I/M/K/R, H113A/N/Q/T/V/F/L/I/M/Y, Y116A/L/I/M/W, D171A/E/N/V/L/I/M/K, S178A/T/V/L/I, A180S/V/L/I/M, D181A/E/N/V/L/I/M, D182A/E/N/V/L/I/M, R183A/K/Q/L/I/M, 1185A/L/F/M, D187A/E/N/V/L/I/M/K, R188A/K/Q/L/I/M, F189A/Y/L/I/M/W, Y190A/F/L/I/M/W, V196A/T/L/I/M, Q200A/N/V/L/I/M, H203A/N/Q/T/V/F/L/I, Y255A/F/L/I/M/W, 1259A/L/V/F/M, D262A/E/N/V/L/I/M, H281A/N/Q/T/V/F/L/I/K, 1284A/L/V/F/M, S285A/T/V/L/I, E288A/D/N/V/L/I/M/K, F292A/Y/L/I/M/W.

### Experimental Screening & Validation

### Expression Constructs

WT CXCR4 with an N-terminal 3×HA-tag, G_{β3}-WT, and GNA15 sub-cloned into pcDNA3.1+ were obtained from the cDNA Resource Center (Bloomsberg, PA). Designed CXCR4 variants and library point mutants were generated by site-directed mutagenesis. BRET fusion constructs for G_{αi1}-91-Rluc8, G_{γ9}-GFP2, and β-arrestin2-Rluc8 were derived from optimized Tru-path constructs³⁹ and sub-cloned into pcDNA3.1+(Genscript Biotech).

### Modified Peptides

Peptides were synthesized with C-terminal amidation (to reduce unwanted charge effects at the carboxy terminus) to generate wild-type and variants of the 17 N-terminal residues of CXCL12 (KPVSLSYRCPCRFFESH) (GenScript Biotech), a peptide known to elicit calcium mobilization and Gᵢ coupling signaling^{19,20}. Lyophilized peptides were stored at −80° C. and resuspended in assay buffer on day of experiment.

### Calcium Mobilization Assays

40,000 HEK 293T cells were transiently transfected with 50 ng HA₃-CXCR4, 10 ng GNA15 in pcDNA3.1+. To equalize receptor surface expression, 75 ng of HA₃-CXCR4 for the Cdes2 Design variant. Cells were first seeded in 100 uL DMEM (Gibco, ref: 41965-039) 10% FBS in a black-walled, clear-bottom 96-well plate coated with poly-lysine (Sigma, P6407-5MG). Directly after cell loading, 50 uL of the mixture containing 0.5 μL Lipofectamine 2000 (Invitrogen, ref: 11668-019) and the DNA was added on top of the cells. The cells were then left to incubate at 37° C., 5% CO₂, 95% relative humidity for 20 h, after which, media was refreshed with 150 uL DMEM+10% FBS. Cells were assayed 48 h post-transfection. Cells were washed with 200 μL FLIPR6 buffer (HBSS+20 mM HEPES, pH 7.4), then incubated at 37° C., 5% CO₂, 95% relative humidity for 2 h in 200 μL dye buffer according to manufacturer's protocol. Just before the assay, 5× concentrated peptide solutions were prepared in FLIPR6 buffer in a V-bottom 96-well plate. After incubation, peptide solutions were added at a rate of 16 μL/s after 30 s and fluorescence changes were monitored for 90 s after addition using microplate reader FlexStation3 (Molecular Devices). The maximum response after correction by the mock-transfected condition was averaged from three replicates. Maximum values were then plotted against selected concentrations and fitted to a sigmoidal curve.

### Receptor Expression—Enzyme Linked Immunosorbent Assay

Receptor expression measured by ELISA was performed in parallel to each experiment in a poly-lysine coated, white-walled, clear-bottom 96-well plate. Cells were fixed with 4% paraformaldehyde (EMS, ref: 15710) in PBS for 15 min at RT and blocked with 2% BSA for 45 min. After that, 45 min incubations, first with an anti-HA antibody (Thermofisher, ref: 26183) at a dilution of 1:500 followed by a second anti-mouse IgG antibody (CST, ref: 7076S) at 1:2000 dilution, were performed. Finally, chemiluminescence was recorded at the FlexStation3 after 10 min incubation with substrate A and B of SuperSignal™ West Pico PLUS kit (Thermofisher, ref: 34577). Data are plotted as maximum values (n=3, SD plotted).

### β-Arrestin2 Recruitment BRET

40,000 HEK 293T cells were transiently transfected with HA₃-CXCR4, β-arrestin2-Rluc8 and rGFP-CVIM in pcDNA3.1+ at a ratio of 3:1:7 respectively. Cells were first seeded in 100 μL DMEM 10% FBS in a poly-lysine coated, white-walled, white-bottom 96-well plate. Directly after cell loading, 50 μL of the mixture containing Lipofectamine 2000 and the DNA was added on top of the cells. The cells were then left to incubate at 37° C., 5% CO₂, 95% relative humidity for 20 h, after which, 150 μL media was refreshed. Cells were assayed 48 h post-transfection. Cells were washed with 150 μL PBS, then 40 μL BRET buffer (HBSS, 0.2% Glucose) was added to each well. Coelenterazine 400a (Cayman Chemical, ref: 16157) was first added at a final concentration of 2.5 μM and BRET ratios were measured once using Mithras² LB 943 plate reader (Berthold). After the first measurement, 40 μL of a 3× concentrated agonist solution was added to each well and BRET ratios were measured for another 45 min using Mithras² LB 943 plate reader. “Buffer” and “no receptor” controls were subtracted from the data. Maximum values were then plotted against selected concentrations to yield a typical sigmoid, dose-response curve. (n=3, SD plotted)

### G_{αi} Dissociation BRET

40,000 HEK 293T cells were transiently transfected with HA₃-CXCR4, G_{αi1}-91-Rluc8, G_{β3}-WT and G_{γ9}-GFP2 in pcDNA3.1+ at a ratio of 10:1:10:5 respectively. Cells were first seeded in 100 μL DMEM 10% FBS in a poly-lysine coated, white-walled, white-bottom 96-well plate. Directly after cell loading, 50 μL of the mixture containing Lipofectamine 2000 and the DNA was added on top of the cells. The cells were then left to incubate at 37° C., 5% CO₂, 95% relative humidity for 20 h, after which, 150 μL media was refreshed. Cells were assayed 48 h post-transfection. Cells were washed with 150 μL PBS, then 40 μL BRET buffer (HBSS, 0.2% Glucose) was added to each well. Coelenterazine 400a was first added at a final concentration of 2.5 μM and BRET ratios were measured once using Mithras² LB 943 plate reader. After the first measurement, 40 μL of a 3× concentrated agonist solution was added to each well and BRET ratios were measured for another 30 min using Mithras² LB 943 plate reader. Mock-transfected controls were subtracted from the data. Maximum values were then plotted against selected concentrations and fitted to a sigmoidal curve. (n=3, SD plotted)

### Full-Length Chemokine Purification

CXCL12 and variants expressed in pMS211 (pET21a-based) construct and purified as previously described⁴⁰. N-terminal His-tag and leader sequence cleaved with enterokinase to produce a final product with correct N-terminus. Final lyophilized protein was resuspended at 1 mg/ml in 0.1% mg/ml BSA. Aliquots were snap frozen and stored at −80° C.

### Generation of Retroviral Vectors

Retroviral constructs encoding CXCR4 constructs were generated using the In-Fusion HD Cloning Kit (Takara, ref. 638933) according to manufacturer's instructions. PCR sequences were amplified by high-fidelity PCR (CloneAmp™ HiFi PCR Premix, ref. 639298). p-SFG retroviral backbone containing an IRES-CD19 reporter gene was linearized by Notl-HF (NEB, ref. R3189S) and Xhol (NEB, ref. R0146S) restriction enzyme digestion (2-3 h at 37° C.). PCR fragments were gel purified from an agarose gel using the QIAquick Gel Extraction Kit (Qiagen, ref. 28706X4). Fragments of interest were assembled using the In-Fusion enzyme mix with the linearized backbone to generate the constructs of interest and transformed into stellar competent cells. Plasmid DNA was purified from minipreps with QIAprep spin Miniprep Kit (Promega), and constructs were verified by sequencing (Microsynth).

### Generation of Retroviral Supernatant

Retroviral supernatant was produced by transient transfection of 293T cells as previously described.⁴¹ In brief, 293T cells at 50% confluency were co-transfected with 1) the RDF plasmid encoding the RD114 envelope, 2) the Peg-Pam plasmid encoding MoMLV gag-pol, and 3) the SFG retroviral plasmid of interest (with LTRs and packaging signals), using GeneJuice transfection reagent (Merck, ref. 70967-3) according to manufacturer's instructions. Retroviral supernatants were harvested after 48 and 72 hours of culture, filtered with 0.45 μM filter (Filtropur S, Sarsdedt ref. 83.1826), snap-frozen on a dry ice/100% ethanol mixture, and then stored at −80° C. until use, or used as fresh supernatant.

Peripheral Blood Mononuclear Cells from Healthy Human Donors

Buffy coats from de-identified healthy human volunteer blood donors were obtained from the Center of Interregional Blood Transfusion SRK Bern (Bern, Switzerland).

### Generation of T Cells Expressing CXCR4

Peripheral blood mononuclear cells (PBMCs) were isolated from buffy coats by density gradient centrifugation (Lymphoprep, StemCell #07851). Polyclonal CD4 and CD8 T cells were activated on plates coated with anti-CD3 (1 mg/ml, Biolegend, ref. 317347, clone: OKT3) and anti-CD28 (1 mg/ml, Biolegend, ref. 302934, clone: CD28.2) antibodies in T cell media (RPMI containing 10% FBS, 2 mM L-Glutamine, 1% Penicillin-Streptomycin) with IL-15 and IL-7 (Miltenyi Biotec, 10 ng/ml each, ref. 130-095-362 and ref. 130-095-765 respectively). The day before transduction, non cell tissue culture treated 24 well plate (Grener Bio one, ref. 662102) was coated with retronectin (Takara Bio, ref. T100B) in PBS (7 μg/ml, 1 ml per well), and incubated overnight at 4° C. Three days after activation, retronectin was removed and plate was blocked with RPMI 10% FBS during 15 min at 37° C. Then, media was removed and retroviral supernatant was centrifuged at 2000 g, 1 h, 32° C. on retronectin coated plates. Retroviral supernatant was gently removed and activated T cell suspension at 0.15×10\hat{ }6 cells/ml was added, and centrifuged at 1000 g, 10 min, 21° C. Cells were incubated at 37° C. 5% CO2, for 3 days. After 48 to 72 hours of transduction, T cells were harvested and further expanded in T cell media containing IL-7 and IL-15. Transduced T cells were positively selected with a PE selection kit (EasySep, ref. 17684) and an anti-HAtag-PE antibody (Biolegend, ref. 901518, clone: 16B12) to enrich for transduced T cells.

### Migration Assays

T cells transduced and selected for CXCR4 variant expression from 3-6 donors were stained with 1 uM Vybrant DiO cell-labeling solution (Thermo, ref. V22886) in serum-free RPMI 1640+GlutaMax. 40,000 cells in 75 uL were seeded in each well of 96-well Boyden chambers with 5.0 μm pores (Corning, ref. 3388)⁴². Reservoirs were filled with 200 uL serum-free RPMI 1640+GlutaMax or supplemented with 100 nM chemokine. The bottom of the attractant reservoir was imaged for migrated cells over the course of 8 hours with a Cytation 5 BioSpa (Biotek) at 37° C. with 5% CO₂. Fluorescent spots were counted over time and compared to the no-chemokine control to calculate the migration index (#migrated cells towards attractant/#migrated cells towards no-attractant). The peak migration index was averaged between 3 technical replicates for each transduced donor per attractant concentration were plotted.

### REFERENCES

1. Quijano-Rubio, A. et al. De novo design of modular and tunable protein biosensors. Nature 1-9 (2021) doi:10.1038/s41586-021-03258-z.
2. Glasgow, A. A. et al. Computational design of a modular protein sense-response system. Science 366, 1024-1028 (2019).
3. London, N., Raveh, B. & Schueler-Furman, O. Peptide docking and structure-based characterization of peptide binding: from knowledge to know-how. Current Opinion in Structural Biology 23, 894-902 (2013).
4. Petsalaki, E. & Russell, R. B. Peptide-mediated interactions in biological systems: new discoveries and applications. Current Opinion in Biotechnology 19, 344-350 (2008).
5. Jumper, J. et al. Highly accurate protein structure prediction with AlphaFold. Nature 1-11 (2021) doi:10.1038/s41586-021-03819-2.
6. Baek, M. et al. Accurate prediction of protein structures and interactions using a three-track neural network. Science (2021) doi:10.1126/science.abj8754.
7. Lei, Y. et al. A deep-learning framework for multi-level peptide-protein interaction prediction. Nat Commun 12, 5465 (2021).
8. Ciemny, M. et al. Protein-peptide docking: opportunities and challenges. Drug Discovery Today 23, 1530-1537 (2018).
9. Alam, N. & Schueler-Furman, O. Modeling Peptide-Protein Structure and Binding Using Monte Carlo Sampling Approaches: Rosetta FlexPepDock and FlexPepBind. in Modeling Peptide-Protein Interactions 139-169 (Humana Press, New York, NY, 2017). doi:10.1007/978-1-4939-6798-8_9.
10. Vignali, D. & Kallikourdis, M. Improving homing in T cell therapy. Cytokine & Growth Factor Reviews 36, 107-116 (2017).
11. Sackstein, R., Schatton, T. & Barthel, S. R. T-lymphocyte homing: an underappreciated yet critical hurdle for successful cancer immunotherapy. Laboratory Investigation 97, 669-697 (2017).
12. Garetto, S., Sardi, C., Morone, D. & Kallikourdis, M. Chemokines and T Cell Trafficking into Tumors: Strategies to Enhance Recruitment of T Cells into Tumors. in Defects in T Cell Trafficking and Resistance to Cancer Immunotherapy 163-177 (Springer, Cham, 2016). doi:10.1007/978-3-319-42223-7_7.
13. Slaney, C. Y., Kershaw, M. H. & Darcy, P. K. Trafficking of T Cells into Tumors. Cancer Res 74, 7168-7174 (2014).
14. Melero, I., Rouzaut, A., Motz, G. T. & Coukos, G. T-Cell and NK-Cell Infiltration into Solid Tumors: A Key Limiting Factor for Efficacious Cancer Immunotherapy. Cancer Discov 4, 522-526 (2014).
15. Surve, C. R., To, J. Y., Malik, S., Kim, M. & Smrcka, A. V. Dynamic regulation of neutrophil polarity and migration by the heterotrimeric G protein subunits Gαi-GTP and Gβγ. Sci. Signal. 9, ra22-ra22 (2016).
16. Martinez-Muñoz, L. et al. Separating Actin-Dependent Chemokine Receptor Nanoclustering from Dimerization Indicates a Role for Clustering in CXCR4 Signaling and Function. Molecular Cell 70, 106-119.e10 (2018).
17. Chandan, N. R., Abraham, S., SenGupta, S., Parent, C. A. & Smrcka, A. V. A network of Gαi signaling partners is revealed by proximity labeling proteomics analysis and includes PDZ-RhoGEF. Science Signaling (2022) doi:10.1126/scisignal.abi9869.
18. Swaney, K. F., Huang, C.-H. & Devreotes, P. N. Eukaryotic Chemotaxis: A Network of Signaling Pathways Controls Motility, Directional Sensing, and Polarity. Annual Review of Biophysics 39, 265-289 (2010).
19. Loetscher, P., Gong, J.-H., Dewald, B., Baggiolini, M. & Clark-Lewis, I. N-terminal Peptides of Stromal Cell-derived Factor-1 with CXC Chemokine Receptor 4 Agonist and Antagonist Activities. J. Biol. Chem. 273, 22279-22283 (1998).
20. Szpakowska, M. et al. Different contributions of chemokine N-terminal features attest to a different ligand binding mode and a bias towards activation of ACKR3/CXCR7 compared with CXCR4 and CXCR3. British Journal of Pharmacology 175, 1419-1438 (2018).
21. Burg, J. S. et al. Structural basis for chemokine recognition and activation of a viral G protein-coupled receptor. Science 347, 1113-1117 (2015).
22. Jaracz-Ros, A. et al. Differential activity and selectivity of N-terminal modified CXCL12 chemokines at the CXCR4 and ACKR3 receptors. Journal of Leukocyte Biology 107, 1123-1135 (2020).
23. Crump, M. P. et al. Solution structure and basis for functional activity of stromal cell-derived factor-1; dissociation of CXCR4 activation from binding and inhibition of HIV-1. The EMBO Journal 16, 6996-7007 (1997).
24. Ziarek, J. J. et al. Structural basis for chemokine recognition by a G protein-coupled receptor and implications for receptor activation. Sci. Signal. 10, eaah5756 (2017).
25. Young, M. et al. Computational design of orthogonal membrane receptor-effector switches for rewiring signaling pathways. PNAS 115, 7051-7056 (2018).
26. Parad is, J. S. et al. Computationally designed GPCR quaternary structures bias signaling pathway activation. 2021.09.23.461493 https://www.biorxiv.org/content/10.1101/2021.09.23.461493v1 (2021) doi:10.1101/2021.09.23.461493.
27. Patriarchi, T. et al. Ultrafast neuronal imaging of dopamine dynamics with designed genetically encoded sensors. Science eaat4422 (2018) doi:10.1126/science.aat4422.
28. Qin, L. et al. Crystal structure of the chemokine receptor CXCR4 in complex with a viral chemokine. Science 347, 1117-1122 (2015).
29. Gabler, F. et al. Protein Sequence Analysis Using the MPI Bioinformatics Toolkit. Current Protocols in Bioinformatics 72, e₁₀₈ (2020).
30. Zimmermann, L. et al. A Completely Reimplemented MPI Bioinformatics Toolkit with a New HHpred Server at its Core. Journal of Molecular Biology 430, 2237-2243 (2018).
31. Wang, C., Bradley, P. & Baker, D. Protein-Protein Docking with Backbone Flexibility. Journal of Molecular Biology 373, 503-519 (2007).
32. Canutescu, A. A. & Dunbrack, R. L. Cyclic coordinate descent: A robotics algorithm for protein loop closure. Protein Science 12, 963-972 (2003).
33. Lee, J. et al. CHARMM-GUI Input Generator for NAMD, GROMACS, AMBER, OpenMM, and CHARMM/OpenMM Simulations Using the CHARMM36 Additive Force Field. J. Chem. Theory Comput. 12, 405-413 (2016).
34. Jo, S., Kim, T., lyer, V. G. & Im, W. CHARMM-GUI: A web-based graphical user interface for CHARMM. Journal of Computational Chemistry 29, 1859-1865 (2008).
35. Páll, S., Abraham, M. J., Kutzner, C., Hess, B. & Lindahl, E. Tackling Exascale Software Challenges in Molecular Dynamics Simulations with GROMACS. in Solving Software Challenges for Exascale (eds. Markidis, S. & Laure, E.) 3-27 (Springer International Publishing, 2015). doi:10.1007/978-3-319-15976-8_1.
36. Abraham, M. J. et al. GROMACS: High performance molecular simulations through multi-level parallelism from laptops to supercomputers. SoftwareX 1-2, 19-25 (2015).
37. Klauda, J. B. et al. Update of the CHARMM All-Atom Additive Force Field for Lipids: Validation on Six Lipid Types. J. Phys. Chem. B 114, 7830-7843 (2010).
38. Chen, K.-Y. M., Keri, D. & Barth, P. Computational design of G Protein-Coupled Receptor allosteric signal transductions. Nature Chemical Biology 16, 77-86 (2020).
39. Olsen, R. H. J. et al. TRUPATH, an open-source biosensor platform for interrogating the GPCR transducerome. Nature Chemical Biology 1-9 (2020) doi:10.1038/s41589-020-0535-8.
40. Ngo, T. et al. Crosslinking-guided geometry of a complete CXC receptor-chemokine complex and the basis of chemokine subfamily selectivity. PLOS Biology 18, e3000656 (2020).
41. Arber, C. et al. Survivin-specific T cell receptor targets tumor but not T cells. J Clin Invest 125, 157-168 (2015).
42. Hanes, M. S. et al. Dual Targeting of the Chemokine Receptors CXCR4 and ACKR3 with Novel Engineered Chemokines. J. Biol. Chem. 290, 22385-22397 (2015).

1. A computer-implemented method for engineering the interaction between a protein and a cognate peptide that are capable of forming a molecular complex, wherein the method comprises
(a) preparing in silico a library of test peptides based on the cognate peptide, and/or the molecular complex of the protein and the cognate peptide; and
(a′) preparing in silico a library of test protein scaffolds based at least on the parts of the protein that participate in forming the molecular complex with the cognate peptide;
(b) docking in silico (i) the library of test peptides onto the library of protein scaffolds by modelling peptide-protein molecular complexes, or (ii) the cognate peptide on the library of protein scaffolds, or the library of test peptides onto the protein or the parts of the protein that participate in forming the molecular complex with the cognate peptide, or the library of test peptides onto the library of protein scaffolds by modelling peptide-protein molecular complexes;
(c) identifying the test peptides in the library and/or the protein scaffolds in the library for which low interface energy peptide-protein molecular complexes can be modelled, preferably by a combination of flexible peptide docking and/or de novo protein structure building;
(d) identifying the interacting amino acids of the test peptides and/or the protein scaffolds in the ensemble of low interface energy modelled peptide-protein molecular complexes of step (c);
(e) selecting and substituting in silico one or more of the interacting amino acids of the peptides and/or the protein scaffolds as identified in step (d) based on the ensemble of low interface energy peptide-protein molecular complexes as identified in step (c); and
(f) generating in silico based on the peptides with substituted amino acid(s) and/or the protein scaffolds with substituted amino acid(s) of (e) an ensemble of peptides with substituted amino acid(s) and/or the protein scaffolds with substituted amino acid(s) for which the lowest interface energy peptide-protein molecular complexes can be modelled, thereby obtaining engineered peptides and/or proteins being capable of forming a molecular complex with engineered interaction characteristics.

2. The method of claim 1, wherein the protein is a receptor or a part of the receptor that is capable of binding to a natural ligand of the receptor and the cognate peptide comprises the site of the natural ligand that binds to the receptor.

3. The method of claim 2, wherein the receptor is a G protein-coupled receptor and the natural ligand of the receptor is a peptide, preferably a chemokine.

4. The method of any one of claims 1 to 3 further comprising
(g) further selecting and substituting in silico one or more of the interacting amino acids of the peptides and/or the protein scaffolds as identified in step (d) based on the ensemble of peptides with substituted amino acid(s) and/or the protein scaffolds with substituted amino acid(s) for which the lowest interface energy peptide-protein molecular complexes can be modelled of (f); and
(h) generating in silico based on the peptides with substituted amino acid(s) and/or the protein scaffolds with substituted amino acid(s) of (g) an ensemble of peptides with substituted amino acid(s) and/or the protein scaffolds with substituted amino acid(s) for which the lowest interface energy peptide-protein molecular complexes can be modelled, thereby obtaining further engineered peptides and/or proteins being capable of forming a molecular complex.

5. The method of any one of claims 1 to 4 further comprising after step (d)
(e′) selecting and substituting in silico selected single amino acids of the interacting amino acids of the peptides and/or the protein scaffolds as identified in (d) based on the ensemble of low interface energy models of peptide-protein molecular complexes as identified in step (c);
(f′) identifying the peptides and/or protein scaffolds with single substituted amino acid for which the lowest interface energy models of peptide-protein molecular complexes can be modelled;
(g′) generating in silico based on the peptides and/or the protein scaffolds as identified in step (f′) and the peptides and/or the protein scaffolds as identified in step (f) and/or (h) engineered peptides and/or proteins that each carry at least one substituted interacting amino acid position as identified in step (e′) and at least one substituted interacting amino acid position as identified in step (f) and/or (h) for which the lowest interface energy models of peptide-protein molecular complexes can be modelled.

6. The method of any one of claims 1 to 5, wherein the one or more interacting amino acids of step (e) and/or (g) are at least two amino acids that can be found within the same domain of the protein or a protein scaffold, preferably in a putative binding pocket of the protein or a protein scaffold.

7. The method of any one of claims 1 to 6, further comprising the production of
(i) the engineered peptides and/or proteins as identified in step (f) or (h) or as generated in step (g′) or peptides; and/or
(ii) peptides or proteins that comprise peptides and/or proteins as identified in step (f) or (h) or as generated in step (g′)
by peptide and/or protein synthesis or site-directed mutagenesis.

8. The method of claim 7 further comprising
(i) validation of at least one synthesized or mutated peptide and/or protein in a functional assay, preferably a cell-based functional assay that allows to monitor the formation of a molecular complex between a protein and a peptide.

9. The method of claim 6 or 7, wherein the method further comprises
(j) combining a synthesized or mutated peptide and/or protein into a molecular complex with another synthesized or mutated protein and/or peptide or the native protein or cognate peptide into a molecular complex;
(k) identifying superagonistic pairs of proteins and peptides; and
(l) optionally further refining the superagonistic pairs of proteins and peptides by substituting one or more of the interacting amino acids of the protein/peptide pairs and the identification of protein/peptide pairs that display and improved superagonistic activity, binding selectivity or binding orthogonality as compared to the superagonistic pairs of proteins and peptides of (k).

10. A variant of a human CXCR4-derived protein
(A) as characterized by an amino acid sequence comprising or consisting of SEQ ID NO: 1, wherein at least two, preferably at least three of (i) to (viii) apply:
(i) amino acid position 37 is any other amino acid than N and is preferably A,
(ii) amino acid position 41 is any other amino acid than L and is preferably A or I, and is most preferably I,
(iii) amino acid position 45 is any other amino acid than Y and is preferably F,
(iv) amino acid position 113 is any other amino acid than H and is preferably A, M, or N, and is most preferably N,
(v) amino acid position 178 is any other amino acid than S and is preferably F or A, and is most preferably A,
(vi) amino acid position 181 is any other amino acid than D and is preferably Q,
(vii) amino acid position 185 is any other amino acid than I and is preferably V, and
(viii) amino acid position 285 is any other amino acid than S and is preferably M,
(B) sharing at least 80% sequence identity with the CXCR4-derived protein of (A), provided that at least two, preferably at least three of (i) to (viii) as defined in (A) apply, or
(C) being selected from an amino acid sequence comprising or consisting of SEQ ID NOs 2 to 6.

11. A variant of a human CXCL12-derived peptide
(A) as characterized by an amino acid sequence comprising or consisting of SEQ ID NO: 7, wherein
(i) amino acid position 3 is any other amino acid than V and is preferably L, F, W, or Y, and is most preferably Y, and/or
(ii) amino acid position 7 is any other amino acid than V and is preferably L,
(B) sharing at least 80% sequence identity with the CXCR4-derived protein of (A), provided that (i) and/or (ii) as defined in (A) apply, or
(C) as selected from an amino acid sequence comprising or consisting of SEQ ID NOs 8 to 10.

12. A nucleic acid molecule, preferably a vector encoding the variant of the human CXCR4-derived protein of claim 10 and/or the variant of the human CXCL12-derived peptide of claim 11.

13. A cell, preferably a lymphocyte and most preferably a T-cell comprising the nucleic acid molecule, preferably the vector of claim 12.

14. A molecular complex comprising the variant of a human CXCR4-derived protein of claim 10 and/or the variant of a human CXCL12-derived peptide of claim 11.

15. A composition, preferably a diagnostic or pharmaceutical composition, or a kit comprising the variant of a human CXCR4-derived protein or claim 10 and/or the variant of a human CXCL12-derived peptide of claim 11.


SEQUENCE LISTING (11 sequences)

SEQ ID NO: 1 - protein, 352 aa, Homo sapiens
  REGION 1..352: note = WT_CXCR4
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 380 aa, synthetic construct
  REGION 1..380: note = 3xHA-WT_CXCR4
MYPYDVPDYAYPYDVPDYAYPYDVPDYADEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - protein, 352 aa, synthetic construct
  REGION 1..352: note = Cdes1
MEGISIYTSDNYTEEMGSGDYDSMKEPCFREENANFAKIFLPTIFSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWIMITEALAFFHCCLNPILYAFLGAKFKTSAQHALTSVSRGSSLKILSKGKRGGHSSVSTESESSSFHSS

SEQ ID NO: 4 - protein, 352 aa, synthetic construct
  REGION 1..352: note = Cdes2
MEGISIYTSDNYTEEMGSGDYDSMKEPCFREENANFAKIFLPTIFSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVFEAQDRYVCDRFYPNDLWVVVFQFQHIMVGLILPGIVILSCYCIIISKLSHSKGHQKRKALKTTVILILAFFACWLPYYIGISIDSFILLEIIKQGCEFENTVHKWIMITEALAFFHCCLNPILYAFLGAKFKTSAQHALTSVSRGSSLKILSKGKRGGHSSVSTESESSSFHSS

SEQ ID NO: 5 - protein, 352 aa, synthetic construct
  REGION 1..352: note = Ldes
MEGISIYTSDNYTEEMGSGDYDSMKEPCFREENANFNKIFIPTIYSIIFLTGIVGNGLVILVMGYQKKLRSMTDKYRLHLSVADLLFVITLPFWAVDAVANWYFGNFLCKAVNVIYTVNLYSSVLILAFISLDRYLAIVHATNSQRPRKLLAEKVVYVGVWIPALLLTIPDFIFANVAEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 6 - protein, 352 aa, synthetic construct
  REGION 1..352: note = CLdes
MEGISIYTSDNYTEEMGSGDYDSMKEPCFREENANFAKIFLPTIFSIIFLTGIVGNGLVILVMGYQKKLRSMTDKYRLHLSVADLLFVITLPFWAVDAVANWYFGNFLCKAVNVIYTVNLYSSVLILAFISLDRYLAIVHATNSQRPRKLLAEKVVYVGVWIPALLLTIPDFIFANVFEAQDRYVCDRFYPNDLWVVVFQFQHIMVGLILPGIVILSCYCIIISKLSHSKGHQKRKALKTTVILILAFFACWLPYYIGISIDSFILLEIIKQGCEFENTVHKWIMITEALAFFHCCLNPILYAFLGAKFKTSAQHALTSVSRGSSLKILSKGKRGGHSSVSTESESSSFHSS

SEQ ID NO: 7 - protein, 72 aa, Homo sapiens
  REGION 1..72: note = WT_CXCL12
KPVSLSYRCPCRFFESHVARANVKHLKILNTPNCALQIVARLKNNNRQVCIDPKLKWIQEYLEKALNKRFKM

SEQ ID NO: 8 - protein, 72 aa, synthetic construct
  REGION 1..72: note = V3Y_CXCL12
KPYSLSYRCPCRFFESHVARANVKHLKILNTPNCALQIVARLKNNNRQVCIDPKLKWIQEYLEKALNKRFKM

SEQ ID NO: 9 - protein, 72 aa, synthetic construct
  REGION 1..72: note = Y7L_CXCL12
KPVSLSLRCPCRFFESHVARANVKHLKILNTPNCALQIVARLKNNNRQVCIDPKLKWIQEYLEKALNKRFKM

SEQ ID NO: 10 - protein, 72 aa, synthetic construct
  REGION 1..72: note = V3Y-Y7L_CXCL12
KPYSLSLRCPCRFFESHVARANVKHLKILNTPNCALQIVARLKNNNRQVCIDPKLKWIQEYLEKALNKRFKM

SEQ ID NO: 11 - protein, 17 aa, Homo sapiens
  REGION 1..17: note = 17 N-terminal residues of CXCL12
KPVSLSYRCPCRFFESH